(19) United States
(12) Patent Application Publication
(10) Pub. No.: US 2025/0224263 A1
(43) Pub. Date: Jul. 10, 2025

(54) SYSTEM FOR ELECTRONIC MEASUREMENT OF MILK IMBIBED BY AN INFANT

(71) Applicant: Kaizen Bio-tech (2011) Ltd., CAESAREA (IL)

(72) Inventors: Haim DAHAN, RAANANA (IL); Abi ZAKAY, TEL AVIV (IL); Adir KAN, NATANYA (IL); Adi TRABELSI, TEL AVIV (IL); Amir RICHTMAN, PARDES HANNA (IL); Oded HADOMI, EVEN YEHUDA (IL); Mario MESCHIANY, TEL AVIV (IL)

(73) Assignee: Kaizen Bio-tech (2011) Ltd., CAESAREA (IL)

(21) Appl. No.: 18/853,612

(22) PCT Filed: Apr. 3, 2023
(86) PCT No.: PCT/IL2023/050356

§ 371 Date: Oct. 2, 2024

(30) Foreign Application Priority Data
Apr. 3, 2022 (IL) 291920

(51) Int. Cl. G01F 1/36; A61J 11/00

(52) U.S. Cl. CPC G01F 1/36; A61J 11/001; A61J 11/006

(57) ABSTRACT

A nipple device for electronically measuring flow of milk drawn by an infant through its feeding orifices. The device measures milk flow from the differential pressure generated by the milk flow through a fluid flow resistor. The resistor may be located in the peripheral region of the base of the nipple device, and milk conveyed to and from the resistor by means of passageways in the base unit. The differential pressure generated across the resistor is measured using pressure sensors located at the periphery. The pressure sensors and associated electronic circuits may be incorporated into a flow measuring head, attachable to the base unit of the device through a standard pair of fluid ports, to which can be attached heads for other measurements of the milk properties. Other implementations provide increased accuracy for nipple devices using the pressure difference across the feeding orifices for the flow measurement.

### FIELD

The present disclosure describes technology related to the field of methods and systems for measuring and monitoring the quantity of milk delivered to an infant, and especially by a nursing mother to the infant.

### BACKGROUND

The desire to determine how much of a nursing mother's milk a baby is actually taking in during a breastfeeding session often arises, especially in the first few weeks of an infant's life. Babies may be inclined to suck on a breast as a soothing mechanism, giving the mothers the impression that their babies are feeding, when in actual fact, they are not. Additionally, some mothers do not have an adequate milk supply, especially before breast-feeding has been well-established, and therefore a hungry infant may suckle on the breast for long periods of time, without receiving an adequate supply of milk. Additionally, some babies appear to fall asleep during nursing, and the mother may not realize that they are in fact still feeding. Similar requirements may also be required of an infant who is being bottle-fed, since, even though the exact quantity of milk being taken by the infant can be measured by weighing the bottle before and after feeding, or by viewing the graduations on the bottle, this measurement does not provide a real-time indication of the flow rate during the feeding itself, other than an estimation of the rate at which the milk level in the bottle moves downward during feeding, which is an approximate method.

Furthermore, besides the sum total of milk imbibed by the infant, and the rate of taking of the milk, there are other characteristics of the feeding session that simple present-day devices do not readily provide, such as feeding patterns encountered as a function of the time progress of the feed, which could provide beneficial and comparative information.

In order to give a measurement or at least an indication of the quantity of milk being supplied to a baby, various measurement systems have been proposed, some complex and requiring electronic measurement attachments, and some simple. One technique is disclosed in the article by S. E. J. Daly et al., entitled “The Determination of Short-Term Breast Volume Changes and the Rate of Synthesis of Human Milk Using Computerized Breast Measurement” published in Exp. Physiology, 77, 79-87 (1992). In this technique, changes in breast volume are traced by computerized imaging of the breast before and after feeding. International Patent Publication No. WO 2006/054287 for “Breast Milk Flow Meter Apparatus and Method” by E. Kolberg et al, disclose a technique in which a volumetric flow sensor is placed inside a silicon nipple cap through which the baby suckles. The milk flow data from the sensor is converted into milk volume data which is displayed to the mother. Such systems generally involve attaching electronic or electromechanical flow meters to a fluid flow passage in order to measure the fluid flow. There are several other systems proposed which use electronic flow measurement modules, attached externally to a milk collection device which fits over the mother's breast, to measure the milk flow. One such system is shown in International Patent Application Publication No. WO 2014/174508 for “Measurement of Nursed Breast Milk” to O. Melamed, which shows an external electronic measurement unit to measure the flow of milk through a mechanical flow meter. Likewise, in US Published Application No. 2018/0147124 for “Non-Intrusive Breast Milk Monitoring” to L. A. Drew, there is also shown an external electronic flow measurement and display unit.

A simpler, non-electronic device, resembling a nipple shield, has been described in U.S. Pat. No. 7,896,835 for “Apparatus and Method for Measuring Fluid Flow to a Suckling Baby”, commonly owned by the present applicant, in which a fraction of the main milk flow is passed through a measurement channel having a significantly higher resistance to fluid flow than the main milk flow channel, and the fluid flow into the measurement channel can be measured, such as by observing the length of the measurement channel that has filled with milk after the feeding session. Since the ratio of the fluid resistance of the two channels is known, the length of milk in the measurement channel can provide a measure of the quantity of milk drawn through the main channel.

In International Patent Publication WO/2022/175833, for “Device for Flow Detection of Mother's Milk”, also commonly owned by the present applicant and co-pending the present application, there is described a dedicated nipple shield device, which enables a simple indication to a nursing mother of the flow of milk from her infant during feeding, without the need for any electronic attachments. Additionally, that application also discloses a multi-task nipple shield device, that can be used for performing a number of alternative functions related to different aspects of a nursing mother's needs. The nipple shield device comprises a universal base unit which is fitted over the mother's breast, and which executes the transfer of the mother's milk from her nipple to the baby's mouth, by means of a passageway which conveys the milk to and from a location remote from the nipple. At this remote location, any of a number of different operational heads can be attached, each type of head being adapted to perform its own dedicated function or functions related to the milk. The remote location includes a standardized pair of fluid flow connection terminals, and the various operational heads have matching standardized fluid flow connectors that may be attached to the remote connectors on the base nipple shield. The base unit of the nipple shield is thus universal, and the particular use made of the device depends on the head attached to the remote terminals of the nipple shield. Heads, generally electronically operated, can be attached for various measurement or indicational functions, such as flow measurement, flow indication, medicine addition, measurement of the suction pattern of the baby, milk quality analysis, detection of markers in the milk indicating illness of the mother, and numerous other functional uses.

In International Patent Publication No. WO 2020/025337 to Coroflo Limited, for “Microsensor-based Breastfeeding Volume Measurement Device”, there is shown a differential pressure measurement module placed within the orifice in the nipple, through which the infant sucks milk, a first pressure sensor being positioned at the inlet port of the orifice, close to the mother's source of the milk, and a second pressure sensor close to the outlet port where the infant is sucking. The differential pressure calculated from measurements of the two sensors, gives an indication of the milk flow in the channel of the feeding orifice.

A disadvantage common to all such systems which rely on measurement of the pressure drop as the milk flows through a measurement path having a significant fluid resistance, is that there is a considerable effort that must be exerted by the infant to obtain a good flow of milk. One of the reasons for the additional effort required by the infant in such nipples having a higher resistance flow path, is because the infant's sucking pattern is not a continuous application of negative sucking pressure applied to the nipple, but rather a pulsating series of separate sucking actions, typically at a rate of approximately 2 per second, in a sinusoidal-shaped pattern. This pulsating pattern of sucking is not to be confused with the breathing breaks, which the infant has to take every few seconds, those few seconds involving several such sucking cycles. This pattern of repetitive negative pressure pulses applied to the nipple results in an oscillatory flow of milk to and from the infant, since at every release of suction by the infant, part of the milk in the infant's mouth flows back through the nipple orifice to the volume surrounding the mother's breast, to be again taken in by the infant at the next sucking action of the cycle. As a result, part of the milk flows cyclically backwards and forwards through the nipple orifice, instead of being imbibed by the infant, and the net flow imbibed by the infant is only part of the milk moved by the infant during every sucking action of the cycle. This additional flow results in an unnecessary expenditure of energy by the infant, and hence, uncertainty on the part of the mother, as to whether the infant is receiving a sufficient quantity of milk during the feeding session. A more relevant potential problem for devices which do measure the flow of the milk consumed by an infant, is that if the flow of milk is not steady, or at least unidirectional, the constant changing of the flow may generate a noise problem for the measurement system, which would perform much more accurately if a steady flow of milk was available for the measurement.

As the benefits of breast-feeding become more widely known, more mothers are breast-feeding than in the past, highlighting the need for a simple but accurate device for measuring a baby's milk intake.

The disclosures of each of the publications mentioned in this section and in other sections of the specification, are hereby incorporated by reference, each in its entirety.

### SUMMARY

The present disclosure provides novel devices and methods that overcome at least some of the disadvantages of prior art systems and methods, for measurement of milk flow to a feeding infant. The presently described nipple devices have dome structures similar to commonly used nipple shields, having an internal milk collection volume between the dome structure and the mother's breast, from which the infant sucks milk through an orifice or orifices in the tip region of the nipple dome structure. The nipple devices use the fact that the flow to the infant through the nipple orifice or orifices, is proportional to the pressure difference between the milk expressed at the mother's breast, and the milk sucked by the infant at the output ends of the orifice or orifices, and the presently described nipple devices use novel structures in order to determine this pressure difference.

The nipple structures incorporate elements which provide a pressure differential measurement enabling the milk flow to be readily measured by a dedicated pressure sensor device. Such a pressure sensor device could be incorporated in a microelectronic chip mounted on the nipple device. The nipple device can be configured for use either as a nipple shield for measurement of milk flow from a mother's breast to the infant, or as the nipple cover for mounting on a feeding bottle.

According to a first implementation of these devices, the nipple device uses the principle that the flow of milk from the inside volume of the nipple device, whether mounted on a mother's breast or mounted on a feeding bottle, is proportional to the pressure difference generated by the nursing infant across the nipple orifice or orifices. The feeding orifice or orifices of the nipple connect the volume on the inside of the dome of the nipple device, with the outside of the dome of the nipple device, which the infant holds in its mouth. The pressure on the outer surface of the nipple dome, and hence at the outer end of the orifice channel or channels of the nipple device, is thus the negative pressure resulting from the sucking of the infant on the nipple dome. The pressure at the inner end of the orifice channel or channels, is equal to the pressure in the volume on the inside of the nipple dome, which, in the case of a nursing mother, is the volume between the mother's breast and the nipple device. In the case of the nipple device for use on a feeding bottle, the pressure on the inside will be that of the contents inside the bottle. The pressures both inside and outside of the flexible layer of the nipple device can vary between atmospheric pressure and a sub-atmospheric pressure, with the pressure in both of the volumes generally returning to the equilibrium atmospheric pressure when the baby pauses to breathe between several pulsating sucking actions. However, at any other instant of time, there will be a difference in pressure between the two ends of the orifice channel or channels, resulting from the infant's sucking, and since the system is always trying to get to a pressure equilibrium state, when the baby applies sub-pressure, the pressure in the baby's mouth and in the space between the nipple dome and the mother's nipple will tend to equalize. Therefore, milk will have to flow from the space between the nipple dome and the mother's breast, through the orifice(s) to the inside to the baby's mouth. Since the diameter of the orifice(s) is known, if the difference in pressure is known, the rate of flow of the milk can be determined. Even if the diameter of the orifice(s) is not known, a simple preliminary calibration procedure can be used to determine the relationship between the measured pressure to the flow rate of the milk through the orifice(s). Integration over time of those varying flow rates, will provide the total flow of milk passing through the orifice or orifices to the infant. The present application describes structures and methods which enable measurement of this pressure difference, and hence, the ability to measure the flow of milk from the mother to the infant.

In its simplest form, a nipple device of the type shown in the present disclosure requires a pair of pressure sensors (or a differential pressure sensor), whose inputs are connected by means of passageways respectively to the two regions of the milk flow whose pressures it is desired to measure. The pressure sensor(s) may be located at the outer peripheral edge of the base layer of the device, such that they are not obscured by the infant's mouth. Using the common nipple shield model, this can be achieved by forming the narrow passageways within the material of the nipple shield, with one of the passageways leading from an opening in the outer surface of the domed protrusion of the device, near its top end, such that it conveys the pressure generated within the mouth of the infant down the passageway to the first pressure sensor input, and the other of the passageways leading from an opening on the inside surface of the domed protrusion of the device, such that it conveys the pressure present within the inner volume of the domed protrusion, to the second pressure sensor input. This inner volume is the space where the milk expelled from the mother's breast collects, before being drawn through the feeding orifice(s) by the infant. So long as the resistance of the orifice(s) remains constant, the measured pressure difference thus provides an indication of the fluid flow rate of the milk from the mother to the infant. Since the passageways are hermetically sealed at their outer ends, there will be a minimal entry of milk into the passageways, the pressure at the milk flows being transferred by the trapped air layer in the passageways to the pressure sensor inputs. The presence of such a trapped air layer in the passageways is significant, since it provides a gaseous barrier to prevent the milk from contacting the pressure sensors themselves, which could cause malfunctions in the long term.

According to another exemplary implementation of the devices and methods of the present disclosure, the material layer of the nipple dome region of the device of the present disclosure incorporates a pair of chambers embedded within the material layer. The use of only one pair of chambers is the simplest implementation, but similar measurement systems could also use more than a pair. These chambers may be disposed at any circumferential position of the nipple dome from which the infant sucks to obtain milk, but advantageously, though not necessarily, each may extend around opposite parts of the circumference of the nipple dome region of the device. The positions of the chambers relative to the centerline of the thickness of the layer of material of the nipple are configured to be different. One of the pair of chambers, herewithin known as the first chamber, is disposed closer to the outer surface of the layer of material of the nipple region of the device, than to the inner surface. The other of the pair of chambers, the second chamber, is disposed closer to the inner surface of the material layer of the nipple region of the device, than to the outer surface. The positioning of the chamber closer to one or the other surface of the nipple layer naturally results in a dividing wall between the chamber and the surface to which it is closer, that is thinner and hence more flexible, than the dividing wall between the chamber and the surface that it is further from. This thinner dividing wall could be considered to be a thin flexible membrane. Thus, each of the chambers has a thinner wall which will flex readily to changes in the pressure on its outer surface, and a thicker wall which can be considered to be a quasi-fixed wall which is regarded as not flexing under changes of the pressure in the volume outside of it. Once the outer wall of a chamber is regarded as being fixed because of the comparative rigidity of that rear wall, the application of an external pressure to the thin opposite wall of the chamber, whether above or below ambient pressure, results in a bowing motion of the thinner wall, either inwardly or outwardly, in accordance with the level of the applied outside pressure. The term “outer” used in this paragraph is intended to relate to the direction in which the thin wall is located relative to the material of the nipple, whether facing the mother's breast, or facing the real “outside” world relative to the mother, where the infant is located.

Thus, for instance, if the pressure outside of the nipple is less than the ambient pressure, such as is the normal situation when the infant is nursing, the thin wall of the chamber exposed to that lower pressure in the infant's mouth, will extend outwards, and the volume within the chamber will increase, and since the chamber is a closed volume, the pressures within it will decrease. Likewise, for the chamber with its thin wall facing towards the mother's breast, a reduced pressure within the volume of the domed nipple structure will cause the thin wall of that chamber to bow away from the wall and into the internal volume of the dome nipple structure, and since that chamber is also a closed body, the pressure within that chamber will decrease accordingly. Consequently, a differential pressure will be generated between the first and the second chambers, since the external sub-pressure generated by the sucking of the infant, causes the thinner outer wall of the first chamber to extend outwards more than the thinner wall between the second chamber and the inside of the nipple dome will extend inwards, resulting in a lower pressure within the first chamber than in the second chamber. This analysis makes the approximation that the rigidity of the thicker wall of each chamber is such that its motion under the influence of the pressure applied outside the thicker wall can be neglected in comparison to any motion of the thinner wall. This difference in pressures generated between the two chambers is proportional to the difference in pressures present between the outside surface of the nipple dome structure, and its inner volume. Since that difference in pressure is essentially equal to the difference in pressure across the milk flow orifice or orifices, and that difference in pressure determines the rate of flow of milk from within the nipple inner volume through the orifice or orifices to the infant's mouth on the outer surface, measurement of that pressure difference will therefore provide a measure of the milk flow to the infant.

An alternative way of viewing the interaction of the chambers with the ambient pressures present at the outer and inner surfaces of the nipple dome structure, is to consider the reduced pressure within the first chamber, generated because of the outward motion of the thin wall of the first chamber, as being proportional to the reduced pressure outside of the nipple dome. At the same time, for the second chamber located closer to the inner surface of the nipple dome structure, the pressure generated within that chamber, because of the inward motion of the thin wall of the second chamber, is proportional to the pressure within the inner volume of the nipple dome structure.

In an alternative structure to that described above, each chamber can be provided with one wall having a greater flexibility than its opposing wall, by manufacturing one of the walls of a more flexible material that that of the opposing wall. Such a structure will fulfill the requirements of the chambers of this application, but may not be so cost effective or simple to manufacture.

The above nipple devices have been described in their simplest form, which is also the most economical way of manufacturing the devices. In this basic implementation of the devices, which show how the differential pressure between these chambers can be used to provide information about the milk flow, there is only a single first chamber and a single second chamber. However, it is to be understood that the use of a single first chamber and a single second chamber is not intended to limit the devices, which could also function if either or both of the first and second chambers comprised more than a single chamber. The chambers have been thuswise claimed, as “at least a first chamber” and “at least a second chamber” respectively, in order to claim structures using multiple chambers for each pressure measurement, including structures which may have a different number of chambers for the pressure measurement on the inner space of the nipple dome structure, and the outer space, within the infant's mouth.

In order to measure the level of the differential pressure, one convenient method is to provide the nipple device with narrow passageways connecting the two chambers to a more remote region, such as the region at the periphery of the nipple base surround area, where a differential pressure measurement may be made between the two passageways. Alternatively, for multiple chambers for the inner and outer pressure measurements, it is to be understood that multiple passageways may be used for each input to the differential pressure sensor, and are intended to be thuswise claimed. That differential pressure measurement is then proportional to a measure of the milk flow passing through the orifice or orifices to the infant's mouth. Such a differential pressure measurement may be made by connecting the two fluid connections of a differential pressure sensor to the passageways from the first chamber and the second chamber. Many such pressure sensors are available, including miniature sensors only a few mm. in size, such that they do not involve a large encumbrance to the use of the nipple device. Such sensors may operate using a piezoresistive or a piezoelectric element, or a silicon-based microchip sensor, typically based on strain gauge technology, or any other physical phenomena, such as are used in many available commercial miniature pressure sensor elements. The output of the differential pressure gauge may be input to a readout monitor, which provides an output proportional to the milk flow. Calibration of the sensor is needed to convert its signal output to a reading which reflects the flow rate. The differential pressure measurement may even be transferred and analyzed by an external device, such as a smart phone.

Throughout the embodiments shown in this disclosure, as an alternative to a single differential pressure sensor, individual pressure sensors may be used, one on the end of each of the two passageways, each feeding their output signals to an electronic difference circuit, which then outputs the measured differential pressure. Likewise, in devices using more than two chambers, the differential pressure measurement may be obtained by measurement of the individual pressures between the ends of multiples of passageways, from each set of chambers. Thus, the pressure in the inner volume of the nipple dome obtained in several passageways from several chambers may be measured, and subtracted from that obtained from chambers measuring the pressure in the infant's mouth at the outer volume of the nipple dome, to provide the differential pressure, based on which the milk flow is determined. Consequently, the measurement of individual pressures, as claimed, is intended to cover pressures obtained from such combinations also.

There are a number of details of the structure of the nipple device which need to be observed, in order to provide accurate measurements of the milk flow. Since the accuracy with which the pressure within each chamber is measured, is dependent on the ordered motion of the thin wall under the effect of the pressure, it is important that any motion imparted to the shape of the nipple dome and hence of the chambers therewithin, by the reduced pressure of the infant's sucking, be reduced to a minimum. If the entire shape of the chambers were to become distorted by the sub-pressure of the infant's sucking, or by the physical motion of the tongue or lips of the infant, the motion of the thin wall, and thence the resulting change in volume of the chamber, would be undeterminable, and the pressure readings would be distorted accordingly. In order to prevent the pressure reading from being inaccurate because of this distortion of the shape of the nipple by the infant's sucking, the chambers may advantageously be positioned near the upper end of the dome of the nipple, where the material layer undergoes less distortion or change of shape, because of the increased inherent strength of the more closely curved region of the dome structure closer to the peak, as compared with the lower and less curved wall of the nipple dome structure. Alternatively, the upper region of the dome of the nipple can be artificially strengthened by using material or thickness providing more rigidity in that region. By those means, the chambers become more protected from externally induced mechanical distortions than otherwise. Furthermore, an analysis of the signals representing the pressures of both chambers, should enable determination as to whether the chamber membrane distortion is due to physical pressure of the tongue or lips of the infant, or if it is due to the real sub-pressure applied. The analysis of the feeding pattern will be able to remove such noises, and differentiate the effects of real pressure from other disturbances.

Additionally, distortion of the shape of the nipple could be directly caused by the lips of the infant, which contact the nipple further down on the walls of the nipple, rather than close to the peak of the dome of the nipple. Therefore, for chambers situated in the upper region of the nipple dome structure, or in a specifically strengthened region of the dome structure, the shape of the nipple at the measurement region is not disturbed by the infant's lips to the same extent as chamber situated further down the dome.

Since the use of an electronic measurement using the nipple device of the present disclosure enables measurement not only of the rate and quantity of milk taken by the infant, but also provides a real-time display of the pattern of the sucking and intake by the infant, this feature provides useful information to the mother regarding the progress of the feeding session, and of the tiredness of the infant.

A further advantage of the real-time determination of the sucking action of the infant is that the sucking sinusoidally-shaped tracking of the infant provides important information as to the health and strength of the baby, at least as far as its sucking ability goes, and comparison of that sinusoid measurement with normal standards provides an indication of the infant's development. Electronic data tracking of feeding sessions also enables the development of the infant to be readily followed over long periods.

As previously mentioned, the electronic nipple device of the present disclosure can also be used as a bottle nipple, thereby converting any bottle into a “smart bottle” in a very simple manner, at substantially lower cost than other methods of measurement of the quantity and rate of milk intake by an infant. The electronic nipple device is more accurate than any methods involving inspection of the level of the milk in the bottle as it goes down during the feeding session, besides the additional advantages available for the electronic nipple device of the present application, especially in the field of determining the temporally dependent pattern of the infant's feeding, and characteristics of the infant's feeding habits related thereto. The other advantages mentioned previously are also applicable to the “smart bottle” nipple, in that not only the quantity and rate of intake can be provided, but also the pattern of the infant's milk intake, and any clinical or development information obtained therefrom.

According to further implementations of the devices of the present application, in order to avoid effects of the mouth or tongue motions of the infant from interfering with the shape or form of the orifice or orifices, and hence with the magnitude of the flow resistance of the orifice or orifices, which would affect the accuracy of the differential pressure measurement, and hence the flow measurement, a number of structural improvements to the simple orifices used in prior devices, are proposed. According to a first implementation, the material in which the orifice or orifices are formed, is connected to the remainder of the dome nipple structure by means of a thinner, and hence more flexible, layer of the nipple material, which enables the region of the orifice(s) itself to remain stiffer than the region surrounding it, such that mechanical forces applied thereto by the infant may cause the orifice region to move or tilt, but will reduce the extent of deformation of the orifice or orifices themselves, thereby better maintaining the flow resistance of the orifice or orifices. This implementation effectively causes the orifice or orifices to “float” relative to the rest of the domed nipple structure, such that forces applied onto the upper extremity of the nipple structure are essentially not transferred to the structural form of the orifice or orifices themselves, or at least their effect on the orifice or orifices is reduced. In a similar manner, the region of the orifice or orifices can be made of a stiffer material than that of the rest of the nipple device, such that it does not undergo the same level of distortion when forces are applied to it, as it would if made of the same material.

According to yet another implementation, the inner side of the domed nipple structure can comprise a thicker region having a number of channels within the thickness of that region, these channels leading to the region around the inner orifice opening, such that the mother's milk can flow to the orifice or orifices in the event that the mother's nipple is in close contact with the inner side of the domed structure, and may otherwise block the orifice or orifices.

In order to avoid any of the above-mentioned interference with the measurement of the milk flow at the orifice(s), according to yet another embodiment of the present disclosure, the milk flow itself, instead of flowing directly from the inner side of the domed nipple structure, through the orifice(s) to the infant's mouth, can be conveyed by means of a fluid transfer passageway, to a location near the outer edge of the device, distant from the protruding domed nipple structure, where a constriction is formed in the flow path, and this constriction acts as the fluid flow resistor across which the differential pressure is measured. After flowing through the flow resistor, the milk is conveyed back through a second fluid transfer passageway, to the outer side of the dome nipple structure through an orifice or orifices, and into the mouth of the infant. In this embodiment, the orifice or orifices do not then have any function as the fluid flow resistor of the device, but act merely as the delivery of the milk to the infant from the nipple device. The passageways may be implemented as more than one passageway in each direction, in order to minimize the resistance to the milk flow between the domed nipple structure and the flow resistor. Pressure sensors are installed either at, or in, the end regions of the hydraulic flow resistor, in order to measure the differential pressure across the resistor, and from this differential pressure, the milk flow rate can be determined. The use of a fluid flow resister at a position remote from the feeding orifice(s) thus enables an accurate measure of the flow rate to be obtained, essentially independently of any interference by the infant with the flow emerging from the feeding orifice or orifices.

As in previously described implementations, contact between the milk flow and the pressure sensors can be prevented by use of a trapped air layer in the passageways between the milk flow itself and the pressure sensors. According to another method of preventing milk from contracting the pressure sensors themselves, a pair of pressure transfer chambers can be used, one for the milk flowing into the flow resistor, and one for the milk flowing out of the flow resistor. Each of the chambers has a flexible diaphragm dividing each chamber into two separate sub-chambers. The first pair of sub-chambers are in contact with the milk, one on the input side and the other on the output side of the flow resistor, while the second pair of sub-chambers are in contact with the pressure sensors themselves, one for the pressure on the input side of the flow resistor and the other for the pressure on the output side of the flow resistor. The flexible diaphragms flex in accordance with the pressure applied to them by the milk flow sub-chambers, thus act as a pressure transfer mechanism, transferring those pressures to the pressure sensors, in the second pair of sub-chambers. Since the milk is prevented by the flexible diaphragms from flowing into the second pair of sub-chambers, the pressure sensors are therefore protected from contact with the milk itself, but do sense the fluid pressures of the milk flow by means of the extension of the flexible diaphragms.

The above mentioned International Patent Publication WO2022/175833 for “Device for Flow Detection of Mother's Milk”, there is shown in FIGS. 10 to 12, various implementations of a multitask device for measuring various properties of mother's milk, in the form of a common nipple shield flexible base unit which fits over the mother's nipple, together with various attachment heads available for various different tasks related to the mother's milk supply. The attachment heads are adapted to plug into a fluid connection port located remotely from the nipple region of the device a number of different attachment heads are shown in FIG. 10 of that publication, the common feature of them being that the connection of the head to the fluid connection port completes the circuit for the milk between the passageway or passageways leading from the mother's milk source on the inner side of the flexible nipple shield, and the passageway or passageways leading back to the feeding orifice(s) providing the milk to the infant. One of the attachment heads disclosed in that publication, is a flow indication head providing an indication to the mother that the baby is receiving a flow of milk through the nipple shield, by displaying the flow in a visually transparent section of tubing. In the same manner, an attachment head is disclosed in the present application, in which the flow of milk is determined by incorporating within the attachment head, a flow resistor together with pressure sensors for determining the differential pressure across the flow resistor, and electronic circuitry for converting this differential pressure to a measure of the milk flow. An electronic display can also be incorporated into the attachment head, or alternately, a wireless connection facility for sending the measured flow rate to a remote device, such as a mobile phone. Additionally, pressure transfer chambers, as described hereinabove, can be built into the head.

The above mentioned devices have been described assuming that the flow of milk to the infant is continuous, and without taking into account any pulsating flow pattern of milk through the nipple orifice or orifices, to the infant. The present application also describes a further novel feature of feeding nipples, which provides a steadier flow of milk to the infant, and reduces the effort required by the infant to feed through the nipple, and which then enables every feeding session to be less strenuous to the infant, and with less anxiety to the mother. This feature can be advantageously applied to the measurement nipples so far described hereinabove, but can also be applied to improve nipples of any type in which the feeding orifices are of limited fluid conductance, such as those nipples in which the pressure drop across the orifice is necessary to enable the determination of the flow through the orifice(s), These improved nipples include a partial area of the nipple protrusion surface which is intended to be within the mouth of the infant, having a greater flexibility than that of the remainder of the nipple. This flexible region can be most readily formed by making the partial area of thinner material or of more pliable material than the rest of the nipple area. The flexible region then acts as a pressure equalizer for the action of the infant's sucking, as will be explained hereinbelow.

With prior art nipples, as the infant sucks, a negative pressure is generated within the infant's mouth, and this negative pressure is transferred to the inside volume of the nipple protrusion, from where, milk accumulated in the space between the mother's breast and the nipple's inside surface, is withdrawn. The flow of milk from the mother to the infant arises because the negative pressure in the infant's mouth generated by the infant's sucking has a higher level of negative pressure, i.e. a more negative absolute pressure, than the negative pressure in that inner volume. In the periods of time between the infant's cyclic sucking actions, the infant releases the sub-pressure in its mouth, and the intra-mouth pressure returns essentially to atmospheric pressure, and since the pressure in the inner space of the nipple is then lower than that in the infant's mouth, milk flows back from the infant's mouth into the accumulation of milk in the inner volume of the nipple. The amount of milk flowing back is proportional to the difference in pressure between the baby's mouth, now closer to atmospheric pressure between sucks, and the inside space of the nipple. In the now described nipples of the present disclosure, the flexible membrane region acts to reduce this difference in pressure by flexing to follow the changes in differential pressure between the infant's mouth and the internal nipple space. Thus, while the infant is in the sucking portion of the pulsating feeding process, the flexible membrane moves outward in the direction of the milk flow towards the lower pressure of the infant's mouth. But when the infant relaxes sucking, the flexible membrane bulges inwards towards the mother's breast, since the relaxation of the infant's sucking results in the pressure in the infant's mouth cavity rising towards atmospheric pressure, generating a higher absolute pressure than in the mother's inner space. This inward membrane movement thereby effectively reduces the volume of the space between the nipple and the mother's breast, resulting in the reduction of the extent of the negative pressure (i.e. raising the absolute pressure) of the accumulated milk therein, and thus reducing the differential pressure across the nipple orifice, and hence reducing the backward flow of milk from the infant to the inside volume of the nipple. The rise in pressure on the mother's side, towards the pressure on the infant's side, occurs more rapidly than would be obtained without the flexible membrane, when the tendency to equalization of the two pressures would be dependent on the rate of flow of the milk though the feeding orifice(s).

An alternative, and more graphic way of viewing this step is to regard the inward bulging membrane as enlarging the volume available for the milk in the infant's mouth, thereby making more room to contain the milk which the infant did not swallow, rather than it returning to the mother's side of the nipple. Once the infant again applies suction, the membrane reverses its bulging profile, and moves in an outward direction, since there is now a more negative pressure on the infant's side of the membrane. The differential pressure across the orifice therefore increases and the resulting flow of milk to the infant then continues. The result of the membrane oscillatory motion is thus to reduce the differential pressure across the orifice when the infant is relaxed in the non-sucking phase of the sucking cycle, and to increase or to maintain the differential pressure across the orifice when the infant begins a sucking action again, such that the net effect of the membrane is to increase the net flow of milk from the mother to the infant, and to reduce the extent of the reverse flow of milk from the infant's mouth back to the inner space of the nipple protrusion. This then achieves the double advantage of reducing the effort required by the infant to nurse, and to smooth out the net flow pattern of the milk from the mother to the infant, thereby reducing the “noise” of the differential pressure measurement necessary to determine the milk flow rate.

The above described methods of providing easier feeding for the infant, and for generally reducing the pulsating nature of the infant feeding process, have been described using one or more areas of the nipple region as the flexible membrane to generate the desired effects arising from changes in the comparative volumes available on either side of the nipple protrusion, as the membrane flexes to and fro with the infant's sucking pattern. In the same way, according to a further embodiment of the devices described in this disclosure, it is possible to achieve the same changes in volume between the mother's and the infant's side of the nipple protrusion, when the infant is feeding, by manufacturing the entire nipple protrusion, of a material having substantially higher flexibility than is accepted in the field. Currently available infant nipples, or flow measurement devices based on infant nipple devices, use a flexible material having a hardness in the region of 50 Shore A, or even somewhat less flexible, in order to provide good resistance of the nipple material to the infant's jaw motions, especially with older infants, and hence good wear qualities and long life to the nipple. According to the presently proposed nipple devices, the entire nipple protrusion is formed of a silicone or other flexible layer having a hardness of 40 Shore A, or even less, such as 35 Shore A or even as flexible as 30 Shore A. This provides less resistance of the nipple protrusion to the changes in pressure generated by the sucking or relaxing of the infant feeding, and hence easier feeding and less pulsation. It may also be advantageous to manufacture the entire nipple device of such a more flexible material, in order to reduce manufacturing costs.

The size of the feeding orifice or orifices should be a compromise between being sufficiently small so that the differential pressure between the two ends of the orifice(s) is sufficiently high for the milk flows expected, to enable accurate measurement by the pressure sensors, yet not so small that the orifice(s) represents such a resistance to the flow of milk to the infant, that the infant cannot feed comfortably. The optimum size or sizes can either be measured experimentally or determined from the size of the orifice or orifices used in nipples in general use. However, it is to be understood that the need to measure the differential pressure does place additional constraints on the upper size of the nipple orifice(s).

In practice, the membrane section can be positioned in any part of the nipple protrusion, whether within the region around the orifice in the dome, or on the upper side wall of the nipple protrusion, on condition that it is situated within the infant's mouth when in use. The area of the pressure sensitive membrane is limited by the need to maintain sufficient strength that the membrane does not rupture when strained beyond the limit for which it was designed.

The entire nipple device can be manufactured in a very low cost and high volume manner, by any suitable polymer forming process. The differential pressure sensing device and its controller can be formed on a single microelectronic substrate, such that the electronic readout unit will not take up an appreciable amount of space on or adjacent to the nipple device.

Since the operation of the devices described in the present application, involve negative or sub-atmospheric pressures generated by the sucking action of the infant, and transferred to the mother's nipple, in order to avoid any lack of clarity about the level of these negative pressures, reference to raising or lowering the negative pressure is understood to mean raising or lowering the extent of the negative pressure, even though the pressures are negative. Thus, for example, a term such as “lowering the negative pressure” is not taken in this disclosure to mean making the absolute level of the negative pressure even lower, but rather that the extent of the negative pressure as expressed in the negative level of the pressure, is lowered, which means raising the absolute pressure.

Although reference is made throughout this application to the mother of the infant as being the supplier of the milk, and is also thuswise claimed, this being the usual situation, it is to be understood that references to the mother are not intended to exclude a woman providing the milk other than the infant's mother, and the disclosure and the claims are not intended to be interpreted as limited to a mother using the device to breast-feed her baby.

Additionally, the orifice through which the infant sucks the milk from the inner volume of the dome of the nipple structure, may be a single opening, or several openings, and reference in this disclosure and in the claims to “an orifice” or to “the orifice”, is intended to be interpreted as the total passageway for milk from inside the nipple to the infant's mouth, whether through a single orifice or through more than one orifice.

Furthermore, it is recognized that a differential pressure measurement can be performed either by use of a dedicated differential pressure sensor, or by two separate pressure sensors with a subtraction circuit to provide an output proportional to the difference in pressure between them. Consequently, in this disclosure, and as claimed, any mention of a differential pressure measurement, or a differential pressure sensor, is intended to include measurements performed either by a single differential measurement probe, or by two separate pressure measurement probes.

The above described nipple devices have been shown in their simplest form, which is also the most economical way of manufacturing the devices, in the sense that, in the basic implementation of the devices, there is only a single flexible membrane for providing the pressure compensation to overcome the tendency for milk to return to the mother's side of the nipple structure. However, it is to be understood that the use of a single flexible membrane is not intended to limit the devices, which could also function if more than one flexible membrane were to be used, provided that they were both positioned in locations that would be essentially within the infant's mouth during feeding. The flexible membrane has thuswise been claimed, as “at least one region of the material of the nipple device, . . . having a higher flexibility than the remaining parts of the nipple device” or “at least one area of the domed protrusion . . . ”. Such claim language, or language similar thereto, is intended to also include claim structures using more than one flexible membrane for providing the reverse milk flow compensation. Furthermore, the term “a single flexible membrane”, may in some embodiments, be understood to relate to the whole of the nipple protrusion of the device.

There is thus provided in accordance with an exemplary implementation of the devices described in this disclosure, a device for monitoring a flow of milk drawn by an infant during feeding, the device comprising:
a nipple device for monitoring flow of milk drawn by an infant during feeding, the device comprising:
a base layer with a domed protrusion having an inner surface defining an inner volume of the domed protrusion and an outer surface, the domed protrusion being adapted for insertion into the mouth of the infant;
a first at least one passageway from the inner volume of the domed protrusion to a region of the base layer remote from the domed protrusion; and
a second at least one passageway from the region of the base layer remote from the domed protrusion to at least one position in the outer surface of the domed protrusion,
wherein the first at least one passageway and the second at least one passageway are fluidly connected at the region of the base layer remote from the domed protrusion, by a section of passageway having a first pressure sensor and a second pressure sensor, such that the differential pressure between the first pressure sensor and a second pressure sensor can be determined.

In such a nipple device, the differential pressure between the first pressure sensor and the second pressure sensor enables determination of the flow of milk from within the inner volume of the domed protrusion to the at least one position in the outer surface of the domed protrusion. Additionally, the first and second sensors may be incorporated in a differential pressure module. This differential pressure module may comprise a subtraction circuit operating between the outputs of the pressure sensors.

In any of the nipple devices described above, the connection between the first at least one passageway and the second at least one passageway has a constricted bore to generate increased fluid flow resistance to the flow of milk therethrough. Furthermore, the milk flow to the infant is determined from the differential pressure measured between the pressure sensors, using a known relationship. The output of the pressure measuring devices also enables the pattern of the infant's ingestion of milk to be determined. Additionally, the base layer of the nipple device may be shaped to be mounted on the breast of a mother providing milk to the infant, or it may be adapted to be mounted on a feeding bottle. Furthermore, the region of the base layer remote from the domed protrusion may be a peripheral region of the base layer of the nipple device.

According to further implementations of the devices of this disclosure, in any of the above described devices, the pressure sensors or the differential pressure module may be located in a separate head adapted to be attached to the periphery of the nipple device through fluid flow ports. In such a case, the separate head may comprise either a display for showing the level of the flow of milk, or a wireless facility for transmitting the flow rate to a remote receiver.

In addition, in any of those above described nipple devices, each of the first and the second at least one passageway is connected to a chamber having a flexible diaphragm dividing its internal volume into two hermetically closed compartments, and the pressure transfer between each of the first and the second at least one passageway and its associated pressure sensor is performed across the flexible diaphragm. In such a device, the first at least one passageway may be connected to a first of its two hermetically closed compartments, and the first pressure sensor may be connected to the second of the two hermetically closed compartments. The second of the two hermetically closed compartments may be filled with a liquid.

In yet further implementations of the nipple devices of the present disclosure, the diameter of the passageways may be selected to be sufficiently small that milk entering the passageway at the pressure generated in the device, does not mix with air already in the passageway. Optimally, the passageway has an internal diameter not exceeding 4 mm.

In accordance with yet further implementations of the presently described devices, there is also provided a nipple device to feed an infant, comprising:
a flexible layer having a domed protrusion region;
at least one orifice in the domed protrusion region of the flexible layer, such that at least one passage is formed connecting an inner volume within the domed protrusion region with an outer surface, enabling a flow of milk from the inner volume of the domed protrusion region outward through the at least one orifice; and
at least one higher flexibility area of the domed protrusion region having a flexibility selected to be higher than that of a material of the remaining area of the domed protrusion region, the area being disposed in a location of the domed protrusion region adapted to fit within the mouth of the infant during feeding.

In such a nipple device, the at least one higher flexibility area flexes inwards or outwards of the domed protrusion region in accordance with a differential pressure between the two opposite sides of the at least one higher flexibility area. Also, the at least one higher flexibility area is located within an area which is adapted to be within the infant's mouth when feeding. Furthermore, the at least one higher flexibility area may be located either in the region of the at least one orifice, or in a position in the wall of the domed protrusion region of the nipple device. Also, the flexing of the at least one higher flexibility area is adapted to reduce a change in the differential pressure between the opposite sides of the at least one higher flexibility area, by reducing the volume of that side of the flexible membrane having the lower pressure and increasing the volume of that side of the flexible membrane having the higher pressure.

The inward flexing of the flexible membrane when the infant relaxes a sucking action, may be adapted to reduce the extent of reverse flow of milk from the mouth of the infant to the inner space of the domed nipple protrusion by the process of enlarging the volume available to the infant for keeping milk within his/her mouth. Alternatively, the outward flexing of the flexible membrane when the infant begins a sucking action, may be adapted to increase the extent of flow of milk from the inner space of the domed nipple protrusion to the mouth of the infant, by enlarging the volume of the inner space of the domed nipple protrusion. Also, the differential pressure sensor unit may be pre-calibrated, such that the differential pressure measured is related to the milk flow through the at least one orifice of the nipple device.

According to yet another implementation of these nipple devices, the differential pressure sensor unit may comprise at least one of:
a single differential pressure sensor; or
a pressure sensor for each set of passageways respectively from the inner surface and the outer surface of the domed protrusion, with a subtraction circuit operating between the outputs of the pressure sensors.

In any such devices, the base layer of the nipple device may be adapted to be mounted on a breast of a mother providing milk to the infant, or it may be adapted to be mounted on a feeding bottle. Furthermore, advantageously, the at least one differential pressure sensor unit may located in a peripheral region of the base layer of the nipple device.

In accordance with yet further implementations of the presently described devices, there is also provided a nipple device to feed an infant, comprising:
a flexible layer having a domed protrusion region; and
at least one orifice in the domed protrusion region of the flexible layer, such that at least one passage is formed connecting an inner volume within the domed protrusion region with an outer surface, enabling a flow of milk from the inner volume of the domed protrusion region outward through the at least one orifice; and
wherein the material of at least the domed protrusion has a hardness of less than 40 Shore A.
   In such a nipple device, changes in pressure on either side of the domed protrusion generate a larger change in volume on the opposite sides of the nipple protrusion, than would be obtained using a material having a higher hardness. Furthermore, the material of at least the domed protrusion may have a hardness of less than 35 Shore A. In any event, the entire flexible layer may comprise material having a hardness of less than 40 Shore A, or it may comprise material having a hardness of less than 35 Shore A.

There is further provided according to further embodiments described in this application, a device to monitor a flow of milk drawn by an infant during feeding, the device comprising:
(i) a flexible layer having a domed nipple region adapted to be disposed in the mouth of the infant, and having at least one orifice connecting an inner surface of the domed nipple region with its outer surface, enabling flow of milk from within the domed nipple region to the mouth of the infant;
(ii) a first chamber formed within the layer in the domed nipple region straddled by a first wall and a second wall opposed to the first wall, in a location that is adapted to be disposed within the mouth of the infant when the infant is feeding on the device, the first wall of increased flexibility disposed adjacent to the outer surface of the domed nipple region, and the second wall disposed adjacent to the inner surface of the domed nipple region;
(iii) a second chamber formed within the layer in the domed nipple region straddled by another first wall and another second wall opposed to the other first wall, having the other first wall of increased flexibility disposed adjacent to the inner surface of the domed nipple region, and the other second wall disposed adjacent to the outer surface of the domed nipple region; and
(iv) passageways connect the first chamber and the second chamber to inputs of a differential pressure measurement unit, such that a differential pressure between a first pressure within the first chamber and a second pressure within the second chamber is determined.
   In such a device, the increased flexibility arises from a thinner first wall than the opposing second wall of its respective chamber. At least one of the first walls may have increased flexibility by being formed of a more flexible material than the opposing second wall of its respective chamber.

Furthermore, the differential pressure measurement unit may be pre-calibrated such that the differential pressure measured is related to the milk flow through the at least one orifice of the device. The differential pressure measured may also determine the milk flow in real time. Additionally or alternatively, the differential pressure measured may be used to determine the feeding pattern of the infant as a function of time.

In any of these embodiments, the first and the second chambers may be disposed at different circumferential positions in the domed nipple region of the device. At least one of the first walls having increased flexibility is in the form of a thin membrane. Furthermore, at least one of the chambers may be disposed in a region of the domed nipple region having higher rigidity than other regions of the domed nipple region, such that the at least one chamber is more resistant to physical disturbance by the infant. The higher rigidity of the region of the domed nipple device may result from the at least one chamber being formed in a material having stiffer properties than other regions of the domed nipple device.

Additionally, in any of these above mentioned devices the differential pressure measurement unit may comprise two pressure sensors with a subtraction circuit operating on the outputs of the two pressure sensors. It may also comprise a microelectronic chip mounted on the device. A control unit may be used, adapted to convert the output of the differential pressure measurement unit to a measure of the milk flow through the device to the infant. The control unit may be adapted to convert the output of the differential pressure measurement unit to determine the feeding pattern of the infant.

The base layer of any of the above described nipple devices may be connected to the flexible layer that is adapted to be mounted on the breast of a mother providing milk to the infant. Alternatively, it may be adapted to be mounted on a feeding bottle. Also, the differential pressure measurement unit may be transferred to a remote system to be displayed or analyzed.

Furthermore, the first chamber may comprise multiple first chambers and the second chamber may comprise multiple second chambers, the device further comprising multiple passageways to connect the multiple first chamber to a first input of the differential pressure measurement unit, and multiple passageways to connect the multiple second chamber to a second input of the differential pressure measurement unit.

According to yet another implementation of such devices, there is disclosed a nipple shield device to determine milk flow drawn by an infant during feeding, the nipple shield device comprising:
a base layer; and
a domed protrusion having a dome layer having an inner surface and an outer surface, the domed protrusion extending from the base layer and having at least one orifice disposed through the domed protrusion, the domed protrusion further comprising:
   a first chamber formed within the dome layer straddled by a first wall disposed at an outer surface with increased flexibility, and a second wall disposed at an inner surface; and
   a second chamber formed within the dome layer straddled by another first wall disposed at the inner surface with increased flexibility, and another second wall disposed at an outer surface,
wherein a differential pressure between a first pressure within the first chamber and a second pressure within the second chamber is determined. Such a nipple shield device, may further comprises a pressure measurement unit that determines the differential pressure. Such a device may further comprise:
(i) a first passageway extending from the first chamber to a first output in the pressure measurement unit; and
(ii) a second passageway extending from the second chamber to a second output in the pressure measurement unit,
wherein the differential pressure is measured between a first pressure within the first chamber, and a second pressure within the second chamber. In any of these devices, the base layer of the nipple device may be adapted to be mounted on a feeding bottle.

According to a last implementation of devices of this disclosure, to feed an infant, such devise may comprise:
a flexible layer having a domed protrusion region;
at least one orifice in the domed protrusion region of the flexible layer, such that at least one passage is formed connecting an inner volume within the domed protrusion region with an outer surface, enabling a flow of milk through the at least one orifice, outward from the inner volume of the domed protrusion region; and
fluid connections of the end regions of the at least one passage to a differential pressure measurement module,
wherein the region of at least one orifice comprises at least one structure for reducing changes in fluid resistance of the at least one passage induced during feeding.

In such a device, the region of the domed protrusion region surrounding the at least one orifice may have a higher flexibility than remaining regions of the domed protrusion region. In such a case, the domed protrusion region surrounding the at least one orifice may have either a thinner thickness or different elastic properties from the remaining regions of the domed protrusion region. Additionally, the inner side of the domed protrusion region surrounding at least one orifice may comprise a region of thickness greater than that of the remaining area, the region having a number of channels within the thickness of that region, these channels leading to the region around the inner orifice opening. Additionally, at least one orifice may has a first inner opening whose ends are fluidly connected to the differential pressure measurement module, and a second outer opening having a larger diameter.

Finally, it is to be understood that references to a differential pressure module or unit or the like, for measurement of the difference between the fluid pressures across the fluid flow resistor, may be understood to relate to separate pressure sensors or to both pressure sensors built into a single unit, but necessarily involving two separate pressure measurements. The terms may thus have been used interchangeably, but should be understood to relate to the same type of measurement.

### BRIEF DESCRIPTION OF THE DRAWINGS

The present invention will be understood and appreciated more fully from the following detailed description, taken in conjunction with the drawings in which:

FIG. 1A shows a schematic representation of the manner in which the nipple devices of the type shown in the present disclosure operate; FIG. 1B shows a cross section of on exemplary implementation of the nipple device shown schematically in FIG. 1A; while FIG. 1C shows a schematic isotopic skeleton drawing of a specific exemplary implementation of nipple device of the type shown in FIG. 1A;

FIG. 2 shows a cross-sectional view of the dome region of the nipple device of FIG. 1C;

FIGS. 3A and 3B are temporal plots of the variation of pressure related to the sinusoidal type of sucking action which the infant performs, FIG. 3A shows the negative pressures inside and outside of the nipple, while FIG. 3B shows the differential pressure across the nipple orifice;

FIGS. 4A and 4B schematically illustrate the functionality of the flexible membrane embodiment of the nipples of this disclosure, to reduce the return flow of milk from the infant towards the mother's side of the nipple;

FIGS. 5A and 5B graphically illustrate the effect of the use of a nipple with a flexible membrane on the pressure cycle shown in the real-life plots of FIG. 3A, while FIGS. 5C and 5D show respectively plots against time of the differential pressure for a conventional nipple device of the present disclosure without the flexible membrane, and for a novel nipple device of the present disclosure with the flexible membrane;

FIGS. 6A and 6B illustrate schematically alternative nipple structures using a flexible membrane to reduce the level of backward flow of milk in the nipple, FIG. 6A showing the flexible membrane in the domed region of the nipple device, and FIG. 6B showing the flexible membrane in a side wall of the nipple device;

FIGS. 7A and 7B illustrate schematically two practical implementations of novel orifice structures, which can be used to avoid effects of external forces, such as from the mouth or tongue motions of the infant, from interfering with the shape or form of the feeding orifice, and hence with the magnitude of the flow resistance of the orifice or orifices;

FIGS. 8A and 8B illustrate schematically a novel structure which can be incorporated on the inner side of the domed nipple protrusion around the feeding orifice, in order to prevent its blocking or partial blocking, by the tip of the mother's nipple;

FIG. 9 shows an implementation of the presently described milk flow measurement devices, in which the components and features required for the measurement of the flow, namely the fluid flow resistor and the pressure sensors, are incorporated into a plug-in head;

FIG. 10 shows a method of ensuring that the pressure sensors are not exposed to contact with the milk flow, by use of a pressure transfer chamber unit in which a flexible diaphragm is used for transferring the pressure within the milk in each flow channel to their respective pressure sensors, without allowing any of the milk to touch the pressure sensors; and

FIG. 11 illustrates an exemplary replaceable flow resistor, suitable for use as part of the orifice through which the infant sucks, the flow resistor being cleanable by virtue of its external flow path.

### DETAILED DESCRIPTION

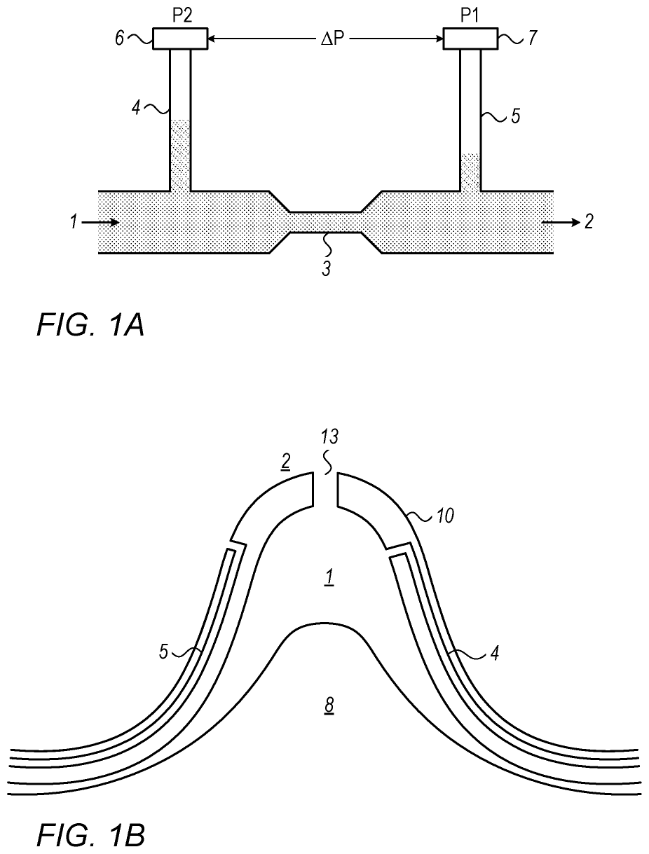

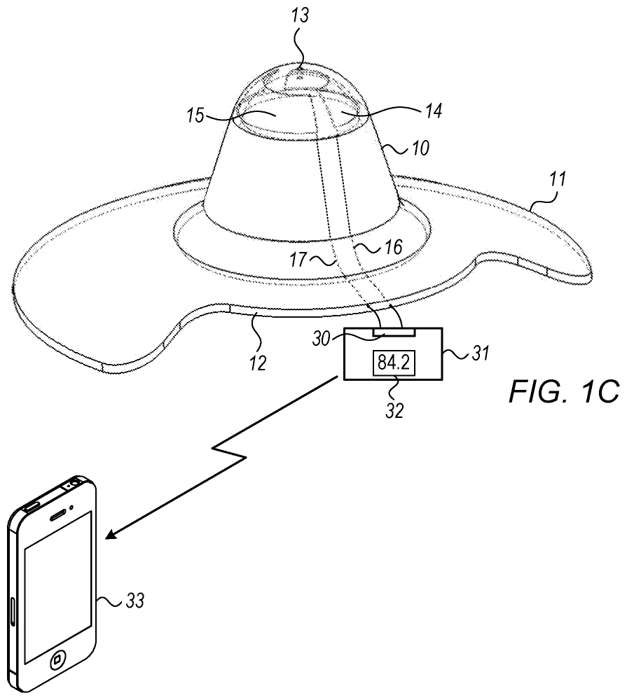

Reference is first made to FIG. 1A, which illustrates a schematic representation of the general manner in which the nipple devices of the type shown in the present disclosure operate. In FIG. 1A, the flow of milk from the mother's breast, represented by region 1, to the infant's mouth, represented by region 2, occurs through the orifice or orifices in the nipple device, which are shown in FIG. 1A as a resistive section 3 of the flow path. Because of the restriction of flow through the resistive section 3, a pressure drop is generated in the flow path between the volume of the mother's side 1 of the device, and that of the infant's side 2 of the device. This pressure difference AP can be measured by two separate pressure sensors 6,7, each connected by means of ports 4, 5, to both sides of the resistive section 3 of the flow path, or by use of a differential pressure sensor (not shown in FIG. 1A). Port 4 measures the pressure P2 on the mother's side, and port 5, the pressure P1 on the infant's side. So long as the resistance of the orifice(s) remains constant, the measured pressure difference AP thus provides an indication of the fluid flow rate of the milk from the mother to the infant. Specific implementations of this model are shown hereinbelow.

In its simplest implementation, the method shown in the schematic device of FIG. 1A, has a particularly useful form, with significant advantages from the point of reusability of the device. The differential pressure sensor, or the two separate pressure sensors, are connected hermetically by means of their respective conduits 4, 5, to the milk flow regions 1, 2 respectively, such that no pressure leakage occurs in the ports or the connecting conduits, thereby impairing the accuracy of the pressure measurements. However, the effect of this is that since the pressure measurement ends of the conduits are closed volumes, the milk only enters the conduits by a very limited amount, conveying the pressure within the conduits to the sensor by means of the trapped air in the conduit. The end of the conduits connected to the differential pressure sensor or the separate pressure sensors 6, 7, and the pressure sensor or sensors themselves, remain free of milk, which is limited to possible small incursions into the conduits close to the milk flow regions. Consequently, the pressure measurement section of the device remains essentially clean of milk, and can be reused without the need to clean it, or with a minimal cleaning procedure which will not cause any damage to the sensitive sensors themselves. The rest of the device, namely the flexible nipple section with the orifice(s) and the conduits conveying the pressures to the connected pressure sensor(s), can then be cleaned by any method deemed sufficiently thorough to provide a nipple safe to use again, including for instance, cleaning in hot or boiling soapy water. The apparatus schematically shown in FIG. 1A, and its method of use, thus enable a milk flow device which is completely reusable, rather than some of the prior art devices, which have to be disposable.

Reference is now made to FIG. 1B, which is a schematic cross section of one exemplary implementation of the nipple device 10 using the concepts shown schematically in FIG. 1A. In FIG. 1B, there is shown a nipple device 10 having an orifice 13 or a number of orifices (not shown in FIG. 1B), in the top region of the domed nipple protrusion of the nipple device. The nipple device is shown as mounted on nipple region of the mother's breast 8, such that the mother's milk collects in the volume 1 between the mother's breast and the inside surface of the domed nipple protrusion. The infant sucks on the outside surface of the domed nipple protrusion, such that the mother's milk flows through the orifice(s) 13 to the infant's mouth 2. Two passages or conduits 4, 5, are formed within the thickness of the flexible material of the domed nipple protrusion 10, one of which 4 opens to the inside volume of the nipple device, where it is in fluid contact with the accumulated mother's milk, and the other of which opens to the external space around the nipple domed structure 10, such that it is positioned within the mouth of the sucking infant when feeding. The passageways lead to connection ports (not shown in FIG. 1B) to locations on the outer edge of the base layer of the nipple device where they can be connected to a differential pressure sensor, or to individual pressure sensors. Though the passageways 4, 5, are shown in FIG. 1B on diametrically opposite sides of the nipple dome, it is to be understood that this is done merely to clearly show both of the passageway in a single cross sectional drawing, and the passageways could advantageously be located in close proximity to each other on one side of the nipple domed protrusion, such that a single differential pressure sensor device could conveniently be connected to their remote ends. It is also to be understood that one or both of the passageways could comprise multiple passageways.

Reference is now made to FIG. 1C, which is a schematic isometric see-through drawing of an exemplary nipple device using the general methods of the basic device described in FIG. 1A, but in which the pressure measurements are performed in a manner completely free of direct contact with the milk. FIG. 1C illustrates the dome region 10 of the nipple device, showing the pressure conveyance passageways which are used to implement the operation of the device. As applied in its simplest implementation, as in FIG. 1B, the pressure conveyance passageways, 16, 17, lead from the upper region of the dome nipple protrusion, one from within the domed nipple protrusion, and one from outside of the protrusion, as shown in FIG. 1B, (where they are denoted by passageways 4 and 5) to a miniature differential pressure measurement control module 31, disposed remotely from the domed nipple protrusion, advantageously at a peripheral region of the device. The module 31 includes a differential pressure sensor 30, which may have a direct read-out display 32 on the module 30 itself, or could send the data to a remote display. The module is configured to use a previous calibration measurement performed on the device to indicate the rate of flow of milk from the mother to the infant.

More complex controllers could be used for outputting a real-time signal proportional to the flow rate such that information can be collected regarding the nature of the infant's feeding habits, the change in feeding action during a feeding session, and, by integrating the signal, the total amount of milk taken by the infant during the whole feeding session. Alternatively, and advantageously, the pressure measurement chip or the control unit can be adapted to transmit its measurements to a remote smart device, such as a mobile phone, where the data can be analyzed and presented. This has the advantage that the control unit 31 can be made much more compact and simpler, since its only function is to export the differential pressure readings to an external control system, where all of the calculations can be executed relating to the milk flow rate, milk quantity or the nature of the feeding process. Furthermore, it has the advantage that the mother or another party can readily read the results of the measurement in real time on a device separate from the nipple device itself. Furthermore, the chip or the control unit or both can be manufactured such that they are transferable from nipple device to nipple device, so that the user only needs one chip or control unit with its electronics, which can be used for many successive nipples.

The nipple device is advantageously formed of a thin layer of flexible material 12, such as a silicone compound, or another suitable flexible polymer, and has a base section 11 from which the dome region 10 extends. The example device shown in FIG. 1C is adapted for use by a nursing mother, who would fit the device over the nipple of her breast, like a conventional nipple shield. A device for use on a feeding bottle (not shown) would typically have a flexible elastically equipped cover section matching the bottle top, instead of the base section 11 of the device shown in FIG. 1C. In common with a conventional nipple shield, the present nipple device has one or more orifices 13 at or around the tip of the domed region of the nipple device, enabling a nursing infant with the domed region located in his/her mouth, to suck the mother's milk from the internal space of the device between the mother's breast and the internal volume of the domed region.

The device shown in FIG. 1C differs from a conventional nipple shield in that it includes two internal chambers 14, 15 formed within the material of the domed protrusion region of the device in its upper region, each connected separately by means of narrow passageways 16, 17, to the pressure measurement control module 31, preferably disposed at an outer part of the base 11 of the device, with each chamber and its narrow passageway being filled with air, and each constituting a closed volume. The chambers are formed close to the tip of the domed region, such that their position is intended to be located within the infant's mouth when the infant is sucking the milk. An advantageous configuration of the chambers is in the form of oppositely positioned chambers around the circumference of the nipple dome. However, the device will be operational with any other suitably positioned chambers.

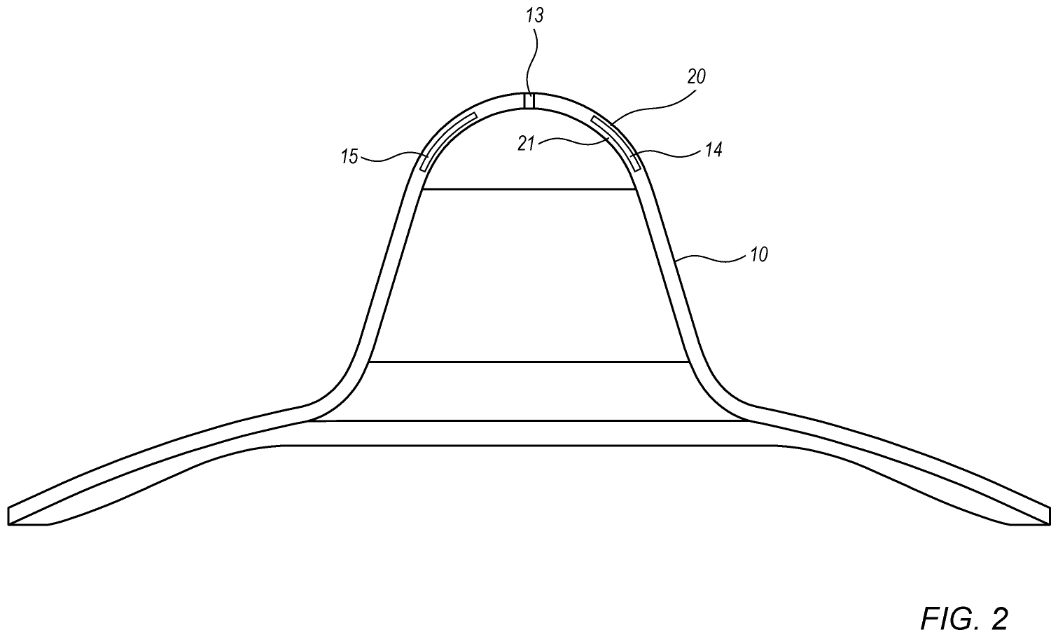

The chambers differ from each other in that they are not equally positioned relative to the centerline of the thickness of the flexible layer in the dome region, as will be more clearly shown in the cross-sectional view of FIG. 2 hereinbelow. The first chamber 14 is positioned closer to the outer surface of the flexible layer than is the second chamber 15, which is positioned closer to the inner volume of the nipple device dome. As a result of this placement within the thickness of the wall of the nipple dome region, the first chamber 14 has a significantly thinner wall with the outside surface of the nipple dome, than the wall with the inner volume of the nipple dome. The second chamber 15, on the other hand, has a significantly thinner wall with the inside surface of the dome than the wall with the outer surface of the nipple dome. The two thin walls may thus be considered as pressure sensitive membranes, which move perpendicularly to the surface of the nipple dome, the extent of the movement being proportional to the pressure applied across the membrane. The thicker walls may be regarded as being essentially stiff static walls compared to the ease of movement of the thin membrane-like walls, such that when the infant sucks on the dome structure in order to obtain milk, the thin-walled membrane of the first chamber 14 moves outward from the dome surface, the extent of the outward movement being proportional to the level of the negative pressure generated by the sucking force of the infant. As the thin-walled membrane moves outwards, since the first chamber is a closed pneumatic system, the pressure of the air inside that system will decrease in a manner proportional to the extent of output motion of the membrane-like wall. The negative pressure generated within the first chamber is thus proportional to the negative pressure generated by the infant's sucking, which is equal to the negative pressure generated at the outer end of the nipple orifice or orifices. In a similar fashion, the thin-walled membrane of the second chamber 15 moves inwards or outwards from the dome surface, the extent and direction of the movement being proportional to the level of the pressure generated within the volume of the nipple. Consequently, that motion of the membrane wall will generate a corresponding pressure within the second chamber 15, such that the pressure within the second chamber reflects the pressure within the inner volume of the nipple, and hence at the inner end of the nipple orifice or orifices. Therefore, the difference in the pressures between the air in the first chamber 14 and the second chamber 15, is a measure of the difference in pressure along the milk orifice or orifices. Since the orifice or orifices have a fixed flow resistance, that difference of pressure along the milk orifice or orifices is directly proportional to the rate of flow of the milk to the infant. As explained hereinabove, the difference in pressures between the two chambers can be readily measured by attaching a differential pressure sensor at the ends of the narrow passageways 16, 17, which convey the pressure levels within the chambers 14, 15, for measurement by the differential pressure sensor 30. Alternatively, separate pressure sensors (not shown in FIG. 1C) may be used to measure each passageway pressure separately, and the difference in reading subtracted to obtain the differential pressure measurement. The volume of the narrow passageways 16, 17, are sufficiently small that they do not significantly affect the level of the pressures measured by the chambers.

The chambers have been described (as will be shown more clearly in FIG. 2 below) as having respectively, a thinner wall towards one of the surfaces of the nipple domed protrusion structure, and a thinner wall towards the other surface of the domed protrusion structure. As explained in the previous paragraph, this structure provides a higher flexibility to the thinner wall than to the opposing wall of the chamber being considered, such that one of the chambers provides an indication of the pressure on one surface of the domed structure, and the other chamber provides an indication of the pressure on the other surface of the domed structure. This method of constructing the chambers is advantageous, since the different walls can be produced simultaneously of the same material as the rest of the nipple device, in a single molding process. However, it is to be understood that the same effect could be provided by making one of the walls of each chamber of a material having a higher flexibility than the material of the opposing wall, such that the essential property of the chambers of this nipple structure, namely having one wall more flexible than the opposing wall, can be achieved thuswise. The important feature of the chambers is that they each have one wall having greater flexibility than the opposing wall, and that one of the chambers has its more flexible wall on the inner surface of the nipple protrusion structure, and the other chamber has its more flexible wall on the outer surface of the nipple protrusion structure.

The device operation has been explained with the chambers 14 close to the orifice or orifices, such that they are located within the mouth of the infant during the feeding session. Since the infant may distort the flexible layer of the dome structure by physical squeezing or pushing of the flexible layer, and this may distort the motion of the membrane-like wall, and hence the pressure level generated within the chamber, the chambers should be located in a region having a higher rigidity than other parts of the nipple dome, so that they are less disturbed by physical forces. As previously stated, the position in the curved upper part of the dome of the nipple has more resistance to distortion than the lower parts of the dome. An increased resistance to distortion can also be achieved by making the material in the upper part of the nipple dome with a higher rigidity than elsewhere on the dome, either by using a stiffer material in that region, or by making the flexible layer thicker in that region. It is of course to be understood that this increased rigidity relates to the thicker wall of the chamber and not to the membrane-like wall, which should maintain the desired flexibility to respond sufficiently to the variable pressure applied to it.

Reference is now made to FIG. 2 which shows a cross-sectional view of the dome region 10 of the nipple device of FIG. 1C, in order to show more clearly, the location of the first pressure measurement chamber 14, and of the second pressure measurement chamber 15, relative to the thickness of the material of the nipple, and a position of the orifice or orifices 13. The narrow passageways 16, 17, shown in FIG. 1C, which convey the pressure levels within the chambers, for measurement by the differential pressure sensor at the edge of the nipple base, are not shown in FIG. 2 to avoid detracting from the purpose of FIG. 2 to show the measurement chamber positions. As is observed, the first chamber 14 is located closer to the outer surface of the dome nipple structure than to the inner surface, such that the wall 20 between the first chamber 14 and the outer surface of the nipple dome structure is significantly thinner than the wall 21 between the first chamber 14 and the inner surface of the nipple dome structure. Consequently, the application of a pressure outside of the thinner wall 20 causes the thinner wall 20 to bulge either outwards or inwards according to the pressure difference applied, while the inner thicker wall 21 is regarded in a first order approximation, to maintain its position without moving. Therefore, the first chamber 14 can be regarded as a measurement device of the externally applied pressure. Conversely, because of the reversed positions of the thinner and thicker walls of the second chamber 15, the second chamber can be regarded as a measurement device of the internal pressure within the nipple dome volume. Therefore, the difference in pressure between the first 14 and second 15 chambers can be used as a measure of the pressure difference across the orifice 13, and hence of the milk flow through the orifice 13.

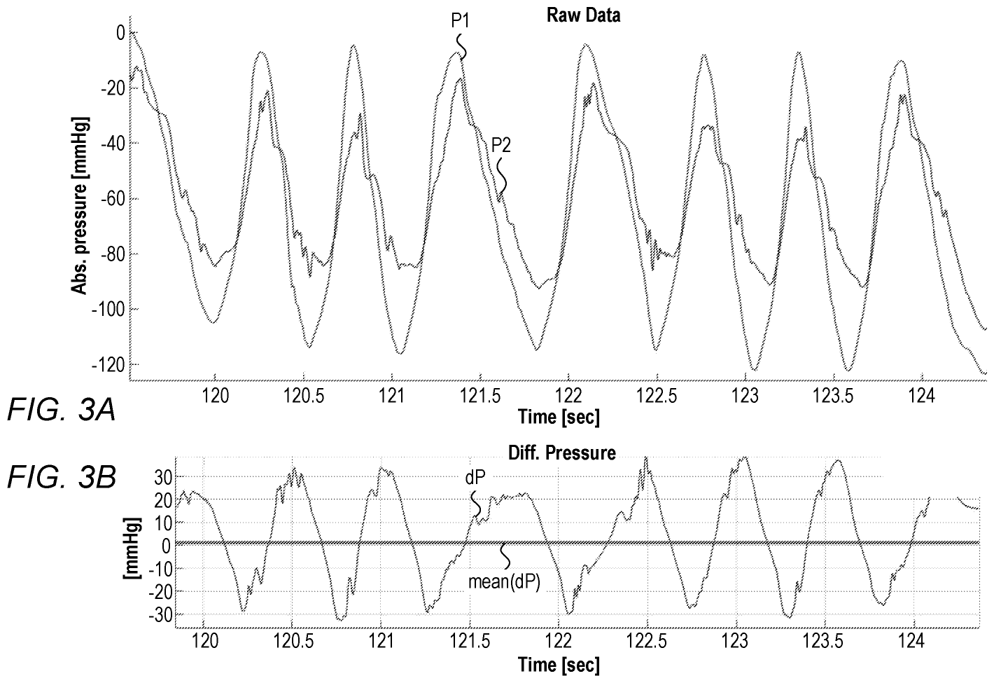

As previously stated, the forward and backward flow of milk through the nipple orifice, caused by the pulsating nature of the infant's sucking, generates a noise level which renders the differential pressure measurement more difficult to perform accurately, and also increases the effort required by the infant to feed from the mother. Reference is now made to FIGS. 3A and 3B, which are plots of the variation with time of the pressure resulting from the sinusoidal, pulsating type of sucking action which the infant performs, on the infant's side of the orifice, and on the mother's side of the feeding orifice. FIG. 3A shows the pressure P1 generated by the infant during the sucking routine, together with the resulting pressure P2 generated on the accumulated milk on the mother's side of the orifice, as a result of the sucking action of the infant on the outside of the nipple. As is observed, the sub-pressures generated on the accumulated milk on the mother's side of the orifice follow the sub-pressures generated by the infant, but over a smaller range, since passage of the milk through the nipple orifice produces a pressure drop. The difference P2−P1, between the two plots shown in FIG. 3A represents the differential pressure which is generated across the orifice, and it is this which determines the flow of the milk through the orifice. At any point of time, the milk flows from the higher absolute pressure to the lower absolute pressure, meaning from points having the lower extent of negative pressure, to points having the higher extent of negative pressure. Thus, on the graph of FIG. 3A, at the bottom of the pressure dips, the milk flows from the mother's side, which is at a higher pressure, to the infant's side, while at the top peaks of the curves, the milk flows back from the infant's side to the mother's side. It is noted that the difference in absolute pressures P2−P1 at the bottom dips of the infant's sinusoidal pressure cycle show a larger difference than the difference in absolute pressures P2−P1 at the top peaks of the infant's pressure cycle. This means that the flow of milk from the inside of the nipple to the infant's mouth is larger than the reverse flow of milk from the infant's mouth back to the accumulated milk within the nipple volume, as is expected from the real-life situation of the feeding process.

Reference is now made to FIG. 3B, where the differential pressure P2−P1, between the infant's side and the mother's side of the nipple, is plotted as a function of time, with the central horizontal line of the graph representing the zero level of differential pressure. As is observed from an inspection of the graph of FIG. 3A, it is clear that the difference P2−P1 is larger at the bottom dips of the curve, representing the point of maximum suction of the infant, than at the peaks of the curves, representing the point of maximum relaxation of the infant's suction. Since the differential pressure, P2−P1 is the driving force for the flow of the milk through the orifice, areas above the zero differential pressure level represent times when there is a forward flow of milk, i.e. from the mother to the infant, while areas below the zero line represent times when there is a reverse flow from the infant back towards the mother's side of the nipple. Therefore, when the integrated area above the zero line is larger than that below the zero line, there is a net flow from the mother to the infant. That is the situation in FIG. 3B, where it is noted that the area within the differential pressure curve above the zero line is larger than the area within the differential pressure curve beneath the zero line, correctly corresponding to the situation that the net flow of milk is from the mother to the infant. It is an object of the additional implementation of the nipple devices of the present disclosure to increase the flow of milk from the mother to the infant as much as possible, and to reduce the reverse flow of milk from the infant towards the mother's side of the nipple as much as possible. This would be represented in FIG. 3B, by a reduction of the area beneath the zero differential pressure line as much as possible.

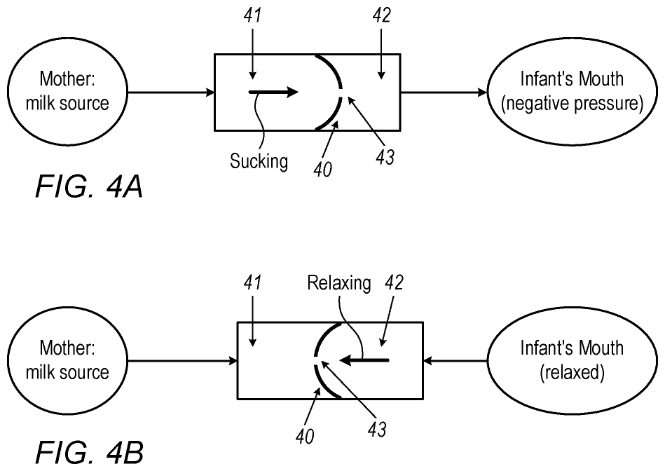

Reference is now made to FIGS. 4A and 4B, which schematically illustrate the functions of a flexible membrane embodiment of the nipples of this disclosure, as described in the Summary section of this disclosure. The drawings show a representation of the flexible membrane 40 dividing the milk regime into two virtual chambers, the left hand chamber 41 representing the mother's side of the nipple, where the mother's milk accumulates, and the right hand chamber 42 representing the infant's mouth cavity, as bounded by the lips of the infant around the nipple. The flexible membrane can be located in the region of the feeding orifice or orifices 43, as shown in the flexible membrane of FIGS. 4A and 4B, but this location is only schematically indicated. In practice, the membrane 40 could be located in any other upper region of the domed nipple protrusion of the device, as a closed flexible partition, dividing the representation of the space between the mother's breast and the inside of the nipple 41, from the infant's mouth cavity 42, and the feeding orifice or orifices can 43 be shown at their conventional location, but other than in the membrane 40. In FIG. 4A, the infant is shown during a sucking phase, and milk is drawn from the mother's side 41 of the nipple through the feeding orifice 43 and to the infant's mouth cavity 42. The more negative pressure in the infant's mouth 42 causes the flexible membrane 40 to bend outwards from the surface of the nipple thereby assisting the passage of milk from the mother's side 41 through the orifice 43. In FIG. 4B, on the other hand, when the infant relaxes his/her sucking action, and the negative pressure in the infant's mouth 42 rises towards atmospheric pressure, there is no longer the pressure pulling the membrane 40 out towards the infant's mouth cavity 41, and the negative pressure still existent on the mother's side 41 of the nipple, causes the membrane 42 reverse direction and to bend into the mother's side 41. This enlarges the available space for the excess milk in the infant's mouth side 42 of the nipple, such that it has less tendency to flow back into the mother's side 41, and at the same time raises the absolute pressure on the mother's side 41 of the nipple, also reducing the fluid tendency of the milk to flow back into the mother's side 41. The results of these functions of the flexible membrane 40 are twofold—firstly that the flow from the mother to the infant when the infant is in the sucking phase of his/her sucking cycle is not impeded, other than the inherent fluid impedance of the limited orifice openings, and secondly, that when the infant stops the sucking phase and relaxes its negative pressure, there is a lower tendency of milk to flow back into the mother's side of the nipple.

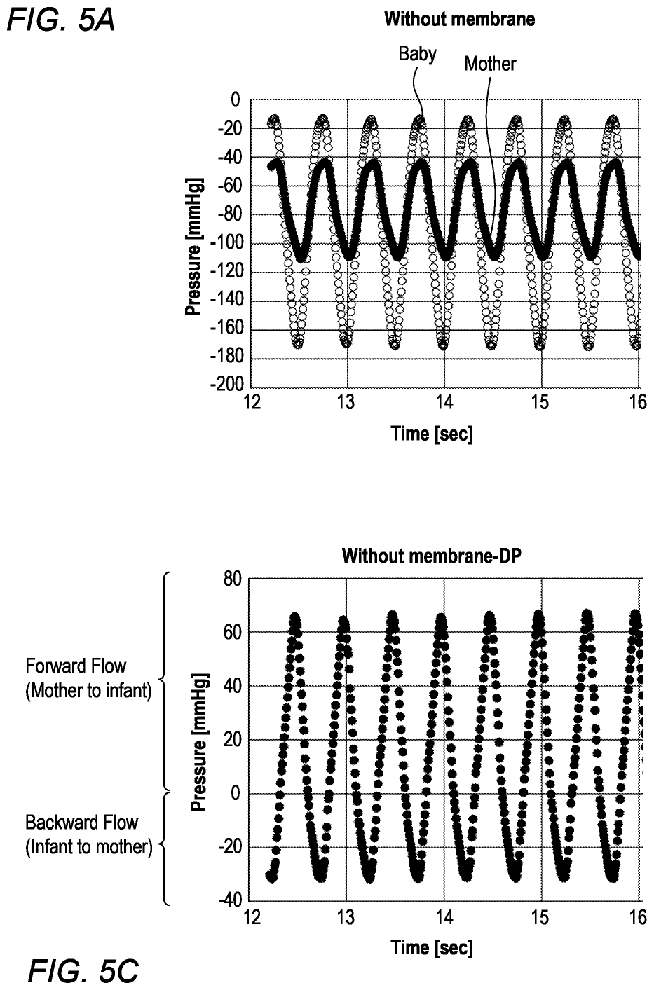

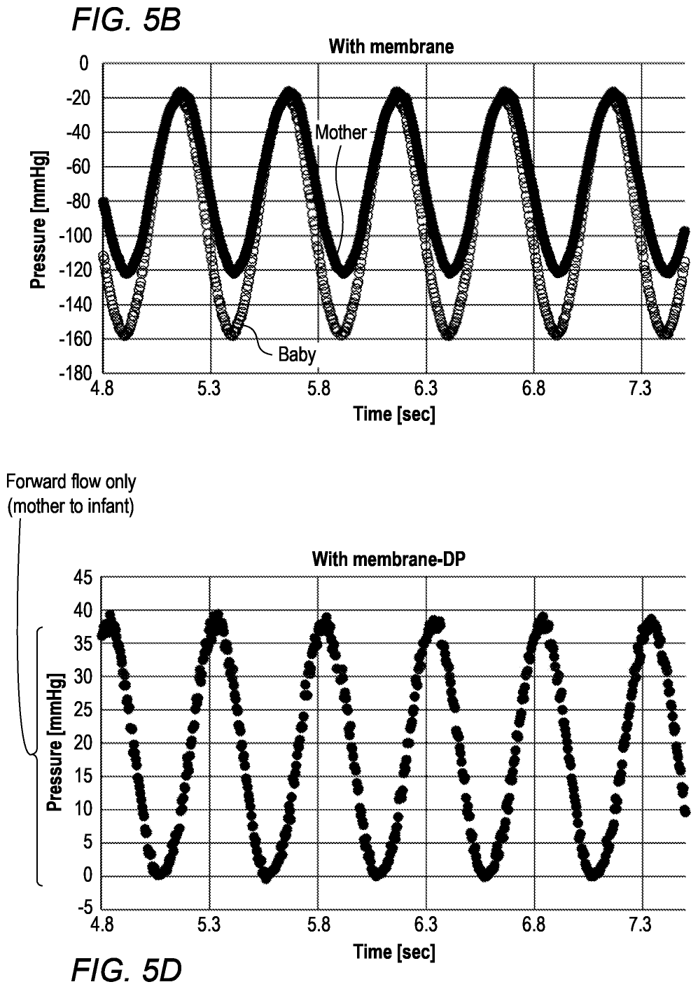

Reference is now made to FIGS. 5A and 5B, which illustrates graphically the effect of the use of a nipple with a flexible membrane on the pressure cycle shown in the real-life plots of FIG. 3A. Firstly, in FIG. 5A, there is shown a plot of the absolute pressures on the infant's side and on the mother's side, resulting from the pulsating sucking action of an infant feeding using a conventional nipple, without the membrane feature of the present disclosure. Each horizontal graduation of the plots represents 1 second. The pressure on the infant's side of the nipple, P1 of FIG. 3A, is denoted by the curve made up of small circles, while the pressure on the mother's side, within the nipple, P2 of FIG. 3A, is shown by the solid curve. As is observed, the infant is sucking at a pulsating rate of 2 Hz. The pressure P1 on the infant's side of the nipple ranges from close to atmospheric pressure, −20 mm of Hg, when the infant has fully relaxed his/her sucking, to a pressure of approximately −170 mm of mercury at the peak of the infant's suction. The resulting pressure range on the mother's side, inside the nipple, ranges from approximately −40 mm Hg, down to −130 mm Hg. Two conclusions can be drawn from these results:
(i) Firstly that the fluid resistance of the orifice(s) to the flow of milk through the orifice(s) connecting the mother's side with the infant's side is sufficiently high that the pressure changes on the mother's side are significantly lower than the pressure changes generated by the infant. In the case of the pressure measurement nipples of the present application, this resistance level has been intentionally selected, rather than a resistance as low as possible, in order to provide sufficient differences in pressure to enable an accurate measurement of the pressure differential, so that the milk flow rate can be accurately measured.
(ii) Secondly, as previously mentioned, it is clear that the large pressure difference between the maximum negative pressure generated by the infant, −170 mm of Hg, and the maximum level of negative pressure, −110 mm of Hg, occurring within the mother's side of the nipple, is indicative of a large flow of milk from the less negative pressure, which is on the mother's side, to the more negative pressure on the infant's side. This difference of pressure is somewhat larger than the difference occurring when the infant is in the relaxation mode, when the flow is from the infant to the mother's side, such that a comparatively small net flow of milk from mother to infant occurs.

FIG. 5B now shows the situation that arises in a nipple which does have the flexible membrane feature of the present disclosure. A significant feature is that the use of the flexible membrane has enabled the pressure within the mother's side to more closely follow that generated by the infant on the infant's side. Thus for instance at the point of maximum sucking relaxation, the pressure on the mother's side goes up to −20 mm. of Hg, which is very close to the pressure of relaxation on the infant's side. Likewise, at the point of maximum suction of the infant, −165 mm of Hg, the negative pressure on the mother's side, −130 mm of Hg, comes much closer than in the nipple without the flexible membrane shown in FIG. 5A. The net result of these two findings is that a significantly smaller proportion of the milk flow through the nipple is returned to the mother's side when the infant relaxes its sucking action. The above described functionality of the flexible membrane can be illustrated by reviewing graphical plots of the differential pressure generated across the milk flow orifice, between the infant's side of the nipple, and the mother's side of the nipple. As previously stated, the differential pressure is defined as the difference between the pressure P1 on the infant's side, and the pressure P2 on the mother's side, i.e. P2−P1. Such graphical plots are now shown in FIGS. 5C and 5D.

FIG. 5C shows a plot against time of the differential pressure for a conventional nipple device without the flexible membrane of the present disclosure. As is observed, the differential pressure ranges from approximately 70 mm of Hg, implying a flow from the mother's side to the infant's side, down to −30 mm of Hg, implying the flow from the infant's side to the mother's side of the nipple, but the important feature of the plot is that a significant part of the integrated pressure plot falls below the zero level of the differential pressure, meaning that there is a significant flow of milk from the infant back to the mother's side of the nipple.

Reference to FIG. 5D, which shows a plot against time of the differential pressure for a nipple device with the novel flexible membrane of the present disclosure, shows that the differential pressure now varies between two much closer pressure levels, over a range of only 40 mm of Hg, which implies that the sub-pressure on the mother's side follows the sub-pressure on the infant's side, which is the driving force for the sub pressure on the mother's side, more closely than in the nipple device without a flexible membrane of FIG. 5C. This immediately suggests that the feeding resistance for the infant is less using the nipple device shown in FIG. 5D with the flexible membrane. However, more important is that the differential pressure, defined as P2−P1, is zero at the maximum relaxation level of the infant, and only moves into positive values as the infant begins the negative pressure of the sucking action. This means that the back flow of milk from the infant back to the mother's side of the nipple has been drastically reduced by use of the flexible membrane of the present application. This feature is also apparent from the plots in FIG. 5B, where it is observed that when the infant is at the peak of the relaxation period, the pressure in the mother's side is essentially equal to the pressure on the infant's side. This result is the reason for the previously mentioned outcome that the feeding resistance for the infant using the nipple with the flexible membrane is less than when nursing from a conventional nipple device of the present disclosure, without the flexible membrane.

FIGS. 5A to 5D thus show how the use of the flexible membrane of the present application significantly improves the ease with which the infant can feed, and also sufficiently reduces the back flow of milk from the infant towards the mother's side of the nipple, such that a more accurate measurement of the sensitive differential pressure measurement can be achieved.

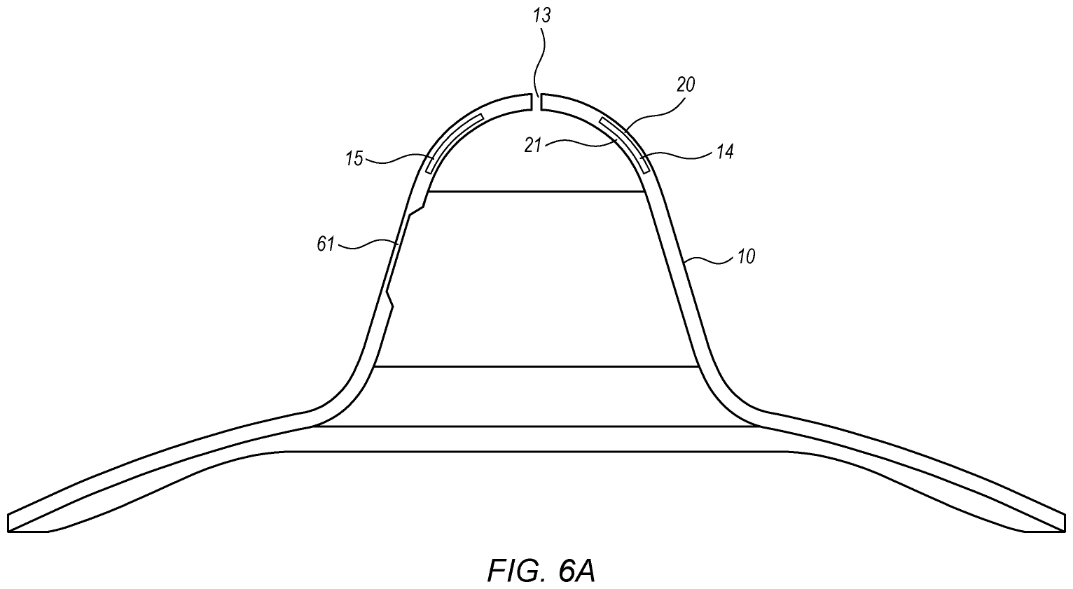

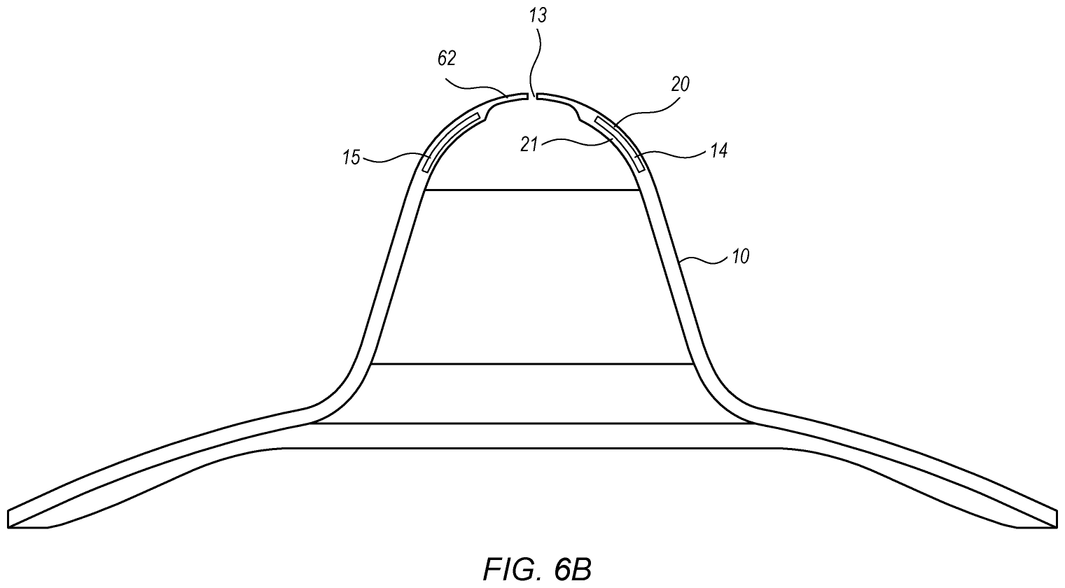

Reference is now made to FIGS. 6A and 6B, which illustrate schematically two practical implementations of the novel use of a flexible membrane in a nipple structure, to achieve the above described advantageous effects. The flexible membrane is shown in FIGS. 6A and 6B, installed on a nipple such as that shown in FIG. 1C and FIG. 2, for measurement of the milk flow by measurement of the differential pressure in the two pressure detection chambers, as explained in connection with FIG. 2. It is to be understood that the flexible membrane feature of the present disclosure, with its concomitant advantages, can also be applied to a conventional prior art nipple without any flow measurement features, but such use may be unnecessary, since in such nipples, the size of the feeding orifice or orifices for can be enlarged to provide as high a fluid conductance path for the infant as is commensurate with a reasonably controlled feeding rate. On the other hand, in the pressure measurement nipple structures of the present disclosure, where the orifice fluid conductance must be limited to ensure that there is a sufficiently large differential pressure across the nipple orifice or orifices, to enable an accurate differential pressure measurement to be obtained, the use of the flexible membrane is very advantageous.

The difference between FIG. 6A and FIG. 6B is only in the location of the flexible membrane in the nipple structure, but the method of operation is the same in the two examples shown. In FIGS. 6A and 6B, the flexible membrane is incorporated into a nipple of the type shown in FIG. 2, and the features of that nipple device are generally labelled as in FIG. 2. In FIG. 6A, the flexible membrane 61 is incorporated into the side wall of the material of the nipple protrusion. It should cover as large a part of the circumference of the nipple protrusion, as possible, to provide maximum change in the volume of the space into which it protrudes, but that area should not be so large that the physical strength of the nipple device is reduced unnecessarily. Furthermore, it should be in a region of the wall that is intended to be within the extent of the inclusion of the infant's mouth when feeding on the nipple, so that it is regarded as being within the infant's mouth cavity. In other words, it must be within the area over which the lips of the infant grip the nipple. This flexible region can be most readily formed by making the area of thinner material or of more pliable material than the rest of the nipple area. Such a thinner region can be readily formed in the molding process of the whole nipple. In FIG. 6B, the flexible membrane 62 is incorporated into the material at the tip of the nipple dome, surrounding the feeding orifice or orifices.

It is to be emphasized that even though the flexible membrane have been shown applied in FIGS. 6A and 6B, to implementations in which the pressure measurements are made using thin-walled chambers, as shown in FIG. 2, this is only one illustration of applications of the flexible membrane in nipple devices, and that the flexible membrane can be installed in nipple devices having any other form of pressure measurement, such as the simple direct measurement embodiment, as shown in FIG. 1B.

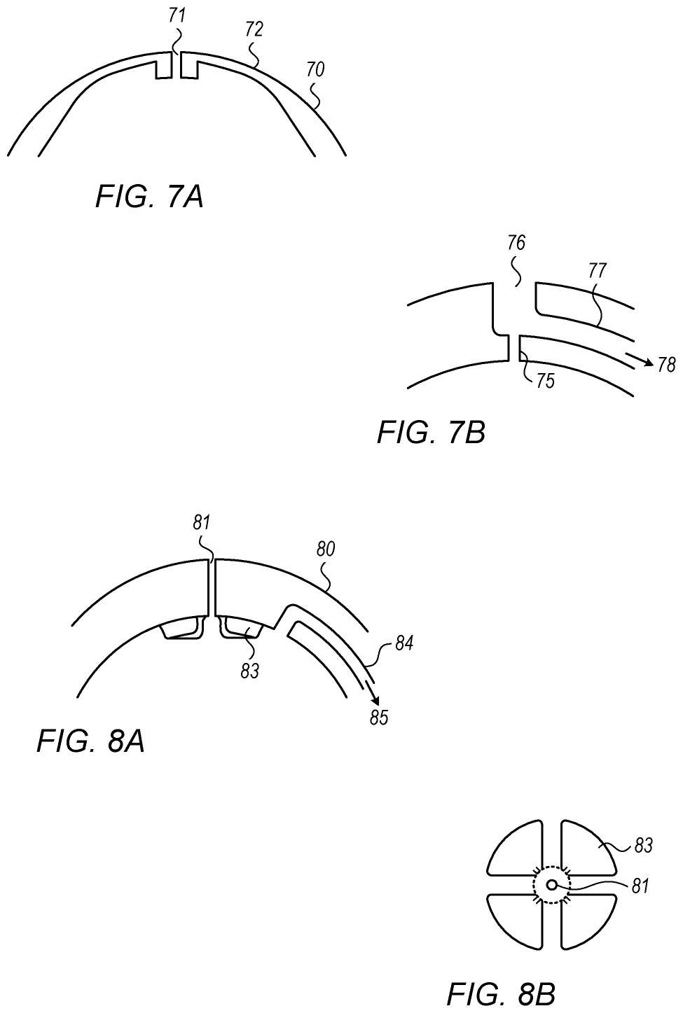

Reference is now made to FIGS. 7A and 7B, which illustrate schematically two practical implementations of novel orifice structures, which can be used to avoid effects of external forces, such as from the mouth or tongue motions of the infant, from interfering with the shape or form of the orifice or orifices, and hence with the magnitude of the flow resistance of the orifice or orifices. In FIG. 7A, there is shown an enlarged cross-section of the tip extremity of the nipple device, with the orifice 71 formed in the nipple device material having its normal thickness, that thickness also being shown down the side 70 of the nipple domed protrusion. The orifice region of the domed nipple protrusion device is connected to the remainder of the device by means of a thinner region 72 of the flexible material, such that the orifice region is flexibly attached to the rest of the device, so that forces applied to the orifice region will cause it to move or change its orientation, but will essentially not deform or compress its shape, thus maintaining the accuracy of the fluid flow resistance through the orifice. A single orifice is shown in FIG. 7A, though it is to be understood that more than one orifice may also be used to provide milk to the infant, and the same requirements that the orifices should not be deformed or compressed apply also to such multiple orifices.

FIG. 7B now shows an alternative or additional method for preventing the tongue of the feeding infant from blocking the orifice of the domed nipple structure. In FIG. 7B, the orifice 75, which acts as the flow resistance for generating the differential pressure for the flow measurement, is shown located at the base of a deeper and slightly wider hole 76. This hole 76 ensures a safe distance between the infant's tongue and the feeding orifice 75, by preventing the tongue from reaching the feeding orifice and possibly blocking it. FIG. 7B also shows the passageway 78 used to convey the pressure of the milk in the infant's mouth, to the pressure sensor for determining the pressure on the infant's side of the flow resistor 75. The passageway 78 should be made comparatively narrow, a typical internal diameter being no more than 4 mm., so that the milk from the baby cannot readily pass down the passageway, mix with the air already trapped within the passageway, and thus prevent the air layer from acting as a gaseous buffer or cushion which is intended to prevent the milk, as far as is possible, from reaching the pressure sensor, which may not take kindly to contact with the milk.

In the same way that the infant's tongue may block the feeding orifice from its outer end, the tip of the mother's nipple may inadvertently block the feeding orifice from its inner end. Reference is now made to FIGS. 8A and 8B, which illustrate schematically a novel structure which can be incorporated on the inner side of the domed nipple protrusion 80, around the feeding orifice 81, in order to prevent its blocking or partial blocking, by the tip of the mother's nipple. On the inner side of the domed nipple protrusion, and surrounding the feeding orifice 81 or orifices, the device is formed with a region of increased thickness 83, having a number of channels 86 within the thickness of that region, these channels leading to the region around the inner orifice opening 81. Consequently, even if one or two of the channels are blocked by the mother's nipple, other channels are open to freely deliver milk from the mother's nipple to the feeding orifice. The increased thickness layer 83 can either be formed in the device material itself, or it can be added as a separately manufactured insert. FIG. 8A also shows the passageway 84 conveying the pressure of the milk 85 on the mother's side of the nipple structure towards the pressure sensor for determining the pressure on the mother's side of the feeding orifice, which acts as the flow resistor.

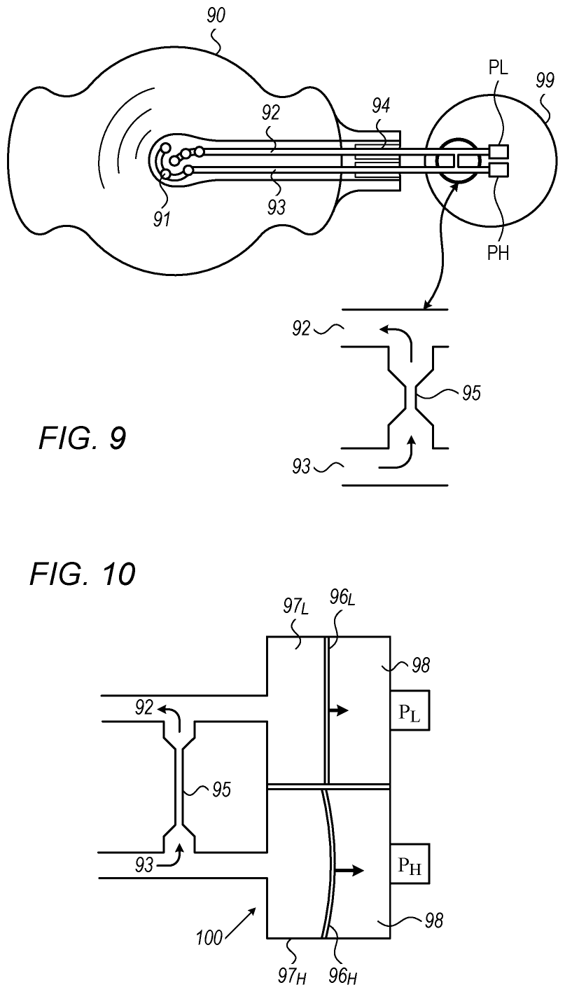

Reference is now made to FIG. 9, which illustrates schematically the implementation of the multi-task milk measurement devices shown in the above mentioned International Patent Publication WO2022/175833, for electronic measurement of milk flow from the mother to the infant. The device comprises a nipple shield flexible base unit 90, and a plug in measurement unit 99, which comprises the differential pressure measurement arrangement. The plug-in measurement unit is connected to the flexible base unit 90 by means of a set of connection ports 94. A pair of passageways are provided connected to the orifices 91 in the top part of the domed protrusion. One of these passageways 93 is for conveying the mother's milk from the inner space of the domed protrusion to the standard attachment ports 94, and a second passageway 92, for conveying the milk back to the feeding orifice in the top part of the domed protrusion, after flow measurement in the plug-in unit 99. The enlarged view at the bottom of FIG. 9 shows the fluid flow resistor 95 connecting the inlet passageway 93 with the return passageway 92. Pressure sensors P_{H} and P_{L}, are shown connected to the inlet passageway 93 and return passageway 92, P_{H} for measuring the pressure of the milk on the input side of the fluid flow resistor 95, and P_{L} for measuring the pressure of the milk after passing through the fluid flow resistor 95. The measurement unit 99 is shown schematically as a circular unit, but it is to be understood that it could be of any other shape. Additionally, the pressure sensors should be connected to electronic circuits (not shown in FIG. 9), for converting the differential pressure measurement into the measured flow level, and an electronic display can also be incorporated into the measurement unit. Alternately, the unit may include a wireless connection facility for sending the measured flow rate to a remote device, such as a mobile phone.

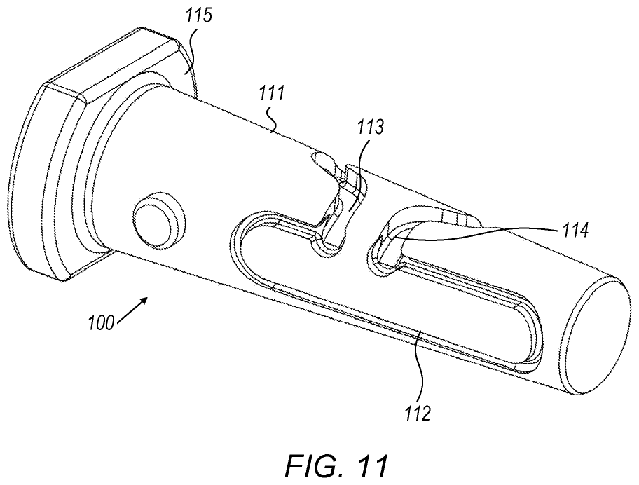

FIG. 9 shows an implementation of the presently described milk flow measurement devices, in which the components and features required for the measurement of the flow, namely the fluid flow resistor and the pressure sensors, are incorporated into a plug-in head 99. According to a further implementation, those components can be incorporated into a self-contained milk flow nipple device, without the advantage of using different plug-in attachment heads for different milk measurement functions. In such an embodiment, the nipple device may comprise only the base unit 90, with the fluid flow resistor 95 mounted within the peripheral skirt region of the device, and not in a separate attachable measurement unit. In any of these devices, the fluid flow resistor 95, shown schematically in FIG. 9 and in FIG. 10, may be implemented as a replaceable resistor, whose function will be further explained hereinbelow in relation to FIG. 11. The replaceable resistor could be mounted either in the plug-in head 99, which is where the flow resistor 95 is shown in FIG. 9, or in the region of the fluid attachment ports 94 in the extremity of the base region of the milk flow measurement device. In either of these cases, a resistor housing may be formed in the region where the resistor is to be inserted, and the resistor can be removably mounted into the base of the device or into the plug-in head, such that the milk flows through it. It can then be removed periodically for cleaning or replacement. Further details are given in relation to FIG. 11 below.

This location of the fluid flow resistor, in the base peripheral region of the device, is an advantageous alternative to using the feeding orifice as the fluid flow resistor with the problems generated thereby, and measuring the pressure drop across the orifice by means of pressure transferring passageways leading to the outer edge of the device base layer, and measuring the differential pressure at that location.

Reference is now made to FIG. 10, which shows an alternative, and more reliable method, of ensuring that the pressure sensors P_{H}, P_{L}, are not exposed to contact with the milk flow. This is achieved by use of a pressure transfer chamber unit 100, in which a flexible diaphragm is used for transferring the pressure within the milk in each flow channel 93, 92, to their respective pressure sensors P_{H}, P_{L}, without allowing any of the milk to touch the pressure sensors. Relating to the inlet flow of the mother's milk in passageway 93, the pressure of the milk is experienced in the sub-chamber 97_{H}, and the comparatively higher pressure of the inlet flow of the mother's milk, causes the diaphragm 96_{H} on the inlet side of the pressure transfer chamber, to deflect outwards from the inlet sub-chamber 97_{H}, transferring a pressure proportional to the level of the inlet pressure to the inlet sub-chamber 98_{H}, where the pressure level is measured by pressure sensor P_{H}. The same process takes place on the outlet side of the milk flow through the fluid flow resistor 95, where the flexible diaphragm 96_{L} transfers a level of pressure proportional to the pressure in the outlet side to the outlet sub chamber 98_{L}, where it is measured by the outlet pressure sensor P_{L}. Since the pressure on the outlet side is considerably lower than that on the inlet side, the flexing of the outlet flexible diaphragm 96_{L} is considerably less than that of the inlet flexible diaphragm, as is shown by the representation of the length of the arrows on the two flexible diaphragms. The sub-chambers 98_{H} and 98_{L}, to which the pressure experienced is transferred, may be advantageously filled with oil or another liquid, increasing accuracy because of the essentially non-compressible nature of liquids compared with leaving those sub-chambers air-filled. The stiffness of the flexible diaphragms should also be comparatively high, such that the flexing is limited, and any non-linear elastic effects are avoided. The use of such a pressure transfer chamber unit 100, ensures that the pressure sensors are therefore protected from contact with the milk itself, but do sense the fluid pressures of the milk flow by means of the extension of the flexible diaphragms.

Reference is now made to FIG. 11, shows another implementation of the features of the present disclosure, in the form of a reusable fluid flow resistor. The need for such a reusable resistor is because in those applications where the pressure drop across the nipple orifice is used in order to determine the flow of milk to the infant, it is important that the resistance to flow of the resistor remains at its planned value. Because the resistor is a precision part, it may be advantageous to have such a reusable fluid flow resistor, rather than a part of a disposable nipple device. Since the milk flow may leave residues of fat and other milk components on the walls of the resistor, the flow resistance will change unless the resistor is cleaned regularly. Since the bore of the previous flow resistors is so small, it is difficult to clean a resistor with an internal bore.

FIG. 11 illustrates an exemplary replaceable flow resistor 110, suitable for use as part of the orifice through which the infant sucks. The flow resistor of FIG. 11 differs from previously used flow resistors in that the flow path of the milk through the resistor is formed on the outer surface 111 of the flow resistor, rather than as an internal bore. The flow resistor is adapted to fit into a dedicated housing which could be part of the orifices 71 of FIG. 7A, or 75 of FIG. 7B or 81 of FIGS. 8A and 8B. The flow resistor 110 slides into the housing until the shoulder 115 of the resistor meets its matching seat in the housing. When properly seated, the regions 113 and 114 are located respectively opposite an input channel in which the milk from the mother flows, and an output channel from which the milk flows to the exit end of the orifice and to the sucking infant. The passageways to the pressure sensors may also be fluidly connected to the regions 113 and 114. The fluid resistor flow path itself 112 is formed on the outside surface 111 of the flow resistor 110. It has a cross section and length such that it provides the resistance to the milk flow that develops a desired pressure difference between its ends that can be readily measured by the differential pressure measurement module, or the individual pressure sensors. The advantage of the external flow resistor of FIG. 11 is that it can be removed at periodic intervals and thoroughly cleaned to maintain the accuracy of its flow resistance. Such a replaceable resistor can also be implemented in the type of plug-in device shown in FIG. 9, where the housing may be advantageously located horizontally across the direction of the fluid passageways, and the flow would be diverted to run into the resistor 95, in the same way as implemented for the removable resistor mounted at the feeding orifice.

Example embodiments are provided so that this disclosure will be thorough, and will fully convey the scope to those who are skilled in the art. Numerous specific details have been set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. Furthermore, it is appreciated by persons skilled in the art that the present invention is not limited by what has been particularly shown and described hereinabove. Rather the scope of the present invention includes both combinations and subcombinations of various features described hereinabove as well as variations and modifications thereto which would occur to a person of skill in the art upon reading the above description and which are not in the prior art.

1. A nipple device for monitoring flow of milk drawn by an infant during feeding, the device comprising:
a base layer with a domed protrusion having an inner surface defining an inner volume of the domed protrusion and an outer surface, the domed protrusion being adapted for insertion into the mouth of the infant;
a first at least one passageway from the inner volume of the domed protrusion to a region of the base layer remote from the domed protrusion; and
a second at least one passageway from the region of the base layer remote from the domed protrusion to at least one position in the outer surface of the domed protrusion,
wherein the first at least one passageway and the second at least one passageway are fluidly connected at the region of the base layer remote from the domed protrusion, by a section of passageway having a first pressure sensor and a second pressure sensor, such that the differential pressure between the first pressure sensor and a second pressure sensor can be determined.

2. The nipple device according to claim 1, wherein the differential pressure between the first pressure sensor and the second pressure sensor enables determination of the flow of milk from within the inner volume of the domed protrusion to the at least one position in the outer surface of the domed protrusion.

3. The nipple device according to either of claims 1 and 2, wherein the first and second sensors are incorporated in a differential pressure module.

4. The nipple device according to any of the previous claims, wherein the differential pressure module comprises a subtraction circuit operating between the outputs of the pressure sensors.

5. The nipple device according to any of the previous claims, wherein the connection between the first at least one passageway and the second at least one passageway has a constricted bore to generate increased fluid flow resistance to the flow of milk therethrough.

6. The nipple device according to any of the previous claims, wherein the milk flow to the infant is determined from the differential pressure measured between the pressure sensors, using a known relationship.

7. The nipple device according any of the previous claims, wherein the base layer of the nipple device is shaped to be mounted on the breast of a mother providing milk to the infant.

8. The nipple device according any of claims 1 to 6, wherein the base layer of the nipple device is adapted to be mounted on a feeding bottle.

9. The nipple device according to any of the previous claims, wherein the region of the base layer remote from the domed protrusion is a peripheral region of the base layer of the nipple device.

10. The nipple device according to any of claims 1 to 8, wherein the pressure sensors or the differential pressure module are located in a separate head adapted to be attached to the periphery of the nipple device through fluid flow ports.

11. The nipple device according to claim 10, wherein the separate head comprises either a display for showing the level of the flow of milk, or a wireless facility for transmitting the flow rate to a remote receiver.

12. The nipple device according to any of the previous claims, wherein each of the first and the second at least one passageway is connected to a chamber having a flexible diaphragm dividing its internal volume into two hermetically closed compartments, and wherein the pressure transfer between each of the first and the second at least one passageway and its associated pressure sensor, is performed across the flexible diaphragm.

13. The nipple device according to claim 12, wherein the first at least one passageway is connected to a first of its two hermetically closed compartments, and the first pressure sensor is connected to the second of the two hermetically closed compartments.

14. The nipple device according to either of claims 11 and 12, wherein the second of the two hermetically closed compartments is filled with a liquid.

15. The nipple device according to any of the previous claims, wherein the diameter of the passageways is selected to be sufficiently small that milk entering the passageway at the pressure generated in the device, does not mix with air already in the passageway.

16. The nipple device according to claim 14, wherein the passageway has an internal diameter not exceeding 4 mm.

17. The nipple device according to any of the previous claims, wherein the output of the pressure measuring devices enables the pattern of the infant's ingestion of milk to be determined.

18. A nipple device to feed an infant, comprising:
a flexible layer having a domed protrusion region;
at least one orifice in the domed protrusion region of the flexible layer, such that at least one passage is formed connecting an inner volume within the domed protrusion region with an outer surface, enabling a flow of milk from the inner volume of the domed protrusion region outward through the at least one orifice; and
at least one higher flexibility area of the domed protrusion region having a flexibility selected to be higher than that of a material of the remaining area of the domed protrusion region, the area being disposed in a location of the domed protrusion region adapted to fit within the mouth of the infant during feeding.

19. The nipple device according to claim 18, wherein the at least one higher flexibility area flexes inwards or outwards of the domed protrusion region in accordance with a differential pressure between the two opposite sides of the at least one higher flexibility area.

20. The nipple device according to either of claim 18 or 19, wherein the at least one higher flexibility area is located within an area which is adapted to be within the infant's mouth when feeding.

21. The nipple device according to claim 20, wherein the at least one higher flexibility area is located either in the region of the at least one orifice, or in a position in the wall of the domed protrusion region of the nipple device.

22. The nipple device according to any of claims 19 to 21, wherein the flexing of the at least one higher flexibility area is adapted to reduce a change in the differential pressure between the opposite sides of the at least one higher flexibility area, by reducing the volume of that side of the flexible membrane having the lower pressure and increasing the volume of that side of the flexible membrane having the higher pressure.

23. The nipple device according to any of claims 19 to 22, wherein the inward flexing of the flexible membrane when the infant relaxes a sucking action, is adapted to reduce the extent of reverse flow of milk from the mouth of the infant to the inner space of the domed nipple protrusion by enlarging the volume available to the infant for keeping milk within his/her mouth.

24. The nipple device according to any of claims 19 to 22, wherein the outward flexing of the flexible membrane when the infant begins a sucking action, is adapted to increase the extent of flow of milk from the inner space of the domed nipple protrusion to the mouth of the infant, by enlarging the volume of the inner space of the domed nipple protrusion.

25. The nipple device according to claim 19, wherein the differential pressure sensor unit is pre-calibrated, such that the differential pressure measured is related to the milk flow through the at least one orifice of the nipple device.

26. The nipple device according to either of claim 18 or 19, wherein the differential pressure sensor unit comprises at least one of:
a single differential pressure sensor; or
a pressure sensor for each set of passageways respectively from the inner surface and the outer surface of the domed protrusion, with a subtraction circuit operating between the outputs of the pressure sensors.

27. The nipple device according any of claims 18 to 26, wherein the base layer of the nipple device is adapted to be mounted on a breast of a mother providing milk to the infant.

28. The nipple device according any of claims 18 to 26, wherein the base layer of the nipple device is adapted to be mounted on a feeding bottle.

29. The nipple device according to any of claims 18 to 26 wherein the at least one differential pressure sensor unit is located in a peripheral region of the base layer of the nipple device.

30. A nipple device to feed an infant, comprising:
a flexible layer having a domed protrusion region; and
at least one orifice in the domed protrusion region of the flexible layer, such that at least one passage is formed connecting an inner volume within the domed protrusion region with an outer surface, enabling a flow of milk from the inner volume of the domed protrusion region outward through the at least one orifice; and
wherein the material of at least the domed protrusion has a hardness of less than 40 Shore A.

31. The nipple device according to claim 30, wherein changes in pressure on either side of the domed protrusion generate a larger change in volume on the opposite sides of the nipple protrusion, than would be obtained using a material having a higher hardness.

32. The nipple device according to either of claim 30 or 31, wherein the material of at least the domed protrusion has a hardness of less than 35 Shore A.

33. The nipple device according to any of claims 30 to 32, wherein the entire flexible layer comprises material having a hardness of less than 40 Shore A.

34. The nipple device according to any of claims 30 to 32, wherein the entire flexible layer comprises material having a hardness of less than 35 Shore A.

35. A device to monitor a flow of milk drawn by an infant during feeding, the device comprising:
a flexible layer having a domed nipple region adapted to be disposed in the mouth of the infant, and having at least one orifice connecting an inner surface of the domed nipple region with its outer surface, enabling flow of milk from within the domed nipple region to the mouth of the infant;
a first chamber formed within the layer in the domed nipple region straddled by a first wall and a second wall opposed to the first wall, in a location that is adapted to be disposed within the mouth of the infant when the infant is feeding on the device, the first wall of increased flexibility disposed adjacent to the outer surface of the domed nipple region, and the second wall disposed adjacent to the inner surface of the domed nipple region;
a second chamber formed within the layer in the domed nipple region straddled by another first wall and another second wall opposed to the other first wall, having the other first wall of increased flexibility disposed adjacent to the inner surface of the domed nipple region, and the other second wall disposed adjacent to the outer surface of the domed nipple region; and
passageways connect the first chamber and the second chamber to inputs of a differential pressure measurement unit, such that a differential pressure between a first pressure within the first chamber and a second pressure within the second chamber is determined.

36. The device according to claim 35, wherein the increased flexibility is a thinner first wall than the opposing second wall of its respective chamber.

37. The device according to claim 35, wherein at least one of the first walls has increased flexibility by being formed of a more flexible material than the opposing second wall of its respective chamber.

38. The device according to any of claims 35 to 37, wherein the differential pressure measurement unit is pre-calibrated such that the differential pressure measured is related to the milk flow through the at least one orifice of the device.

39. The device according to claim 38, wherein the differential pressure measured determines the milk flow in real time.

40. The device according to claim 38, wherein the differential pressure measured determines the feeding pattern of the infant as a function of time.

41. The device according to any of claims 35 to 40, wherein the first and the second chambers are disposed at different circumferential positions in the domed nipple region of the device.

42. The device according to any of claims 35 to 41, wherein at least one of the first walls having increased flexibility is in the form of a thin membrane.

43. The device according to any of claims 35 to 42, wherein at least one of the chambers is disposed in a region of the domed nipple region having higher rigidity than other regions of the domed nipple region, such that the at least one chamber is more resistant to physical disturbance by the infant.

44. The device according to claim 43, wherein the higher rigidity of the region of the domed nipple device results from the at least one chamber being formed in a material having stiffer properties than other regions of the domed nipple device.

45. The device according to any of claims 35 to 44, wherein the differential pressure measurement unit comprises two pressure sensors with a subtraction circuit operating on the outputs of the two pressure sensors.

46. The device according to any of claims 35 to 45, wherein the differential pressure measurement unit comprises a microelectronic chip mounted on the device.

47. The device according to any of claims 35 to 46, further comprising a control unit adapted to convert the output of the differential pressure measurement unit to a measure of the milk flow through the device to the infant.

48. The device according to any of claims 35 to 47, further comprising a control unit adapted to convert the output of the differential pressure measurement unit to determine the feeding pattern of the infant.

49. The device according to any of the claims 35 to 48, having a base layer connected to the flexible layer that is adapted to be mounted on the breast of a mother providing milk to the infant.

50. The device according to claim 38, wherein the differential pressure measurement unit is transferred to a remote system to be displayed or analyzed.

51. The device according to any of claims 35 to 50, wherein the first chamber may comprise multiple first chambers and the second chamber may comprise multiple second chambers, the device further comprising multiple passageways to connect the multiple first chamber to a first input of the differential pressure measurement unit, and multiple passageways to connect the multiple second chamber to a second input of the differential pressure measurement unit.

52. The nipple device according any of claims 35 to 51, wherein the flexible layer of the nipple device is adapted to be mounted on a feeding bottle.

53. A nipple shield device to determine milk flow drawn by an infant during feeding, the nipple shield device comprising:
a base layer; and
a domed protrusion having a dome layer having an inner surface and an outer surface, the domed protrusion extends from the base layer and has at least one orifice disposed through the domed protrusion, the domed protrusion further comprising:
a first chamber formed within the dome layer straddled by a first wall disposed at an outer surface with increased flexibility, and a second wall disposed at an inner surface; and
a second chamber formed within the dome layer straddled by another first wall disposed at the inner surface with increased flexibility, and another second wall disposed at an outer surface, wherein a differential pressure between a first pressure within the first chamber and a second pressure within the second chamber is determined.

54. The nipple shield device of claim 53, wherein the nipple shield device further comprises a pressure measurement unit that determines the differential pressure.

55. The nipple shield device of claim 54, wherein the nipple shield device further comprises:
a first passageway extending from the first chamber to a first output in the pressure measurement unit; and
a second passageway extending from the second chamber to a second output in the pressure measurement unit,
wherein the differential pressure is measured between a first pressure within the first chamber, and a second pressure within the second chamber.

56. The nipple device according any of claims 53 to 55, wherein the base layer of the nipple device is adapted to be mounted on a feeding bottle.

57. A nipple device to feed an infant, comprising:
a flexible layer having a domed protrusion region;
at least one orifice in the domed protrusion region of the flexible layer, such that at least one passage is formed connecting an inner volume within the domed protrusion region with an outer surface, enabling a flow of milk through the at least one orifice, outward from the inner volume of the domed protrusion region; and
fluid connections of the end regions of the at least one passage to a differential pressure measurement module,
wherein the region of at least one orifice comprises at least one structure for reducing changes in fluid resistance of the at least one passage induced during feeding.

58. The nipple device according to claim 57, wherein the region of the domed protrusion region surrounding the at least one orifice has a higher flexibility than remaining regions of the domed protrusion region.

59. The nipple device according to claim 58, wherein the domed protrusion region surrounding the at least one orifice has either a thinner thickness or different elastic properties from the remaining regions of the domed protrusion region.

60. The nipple device according to claim 57, wherein the inner side of the domed protrusion region surrounding at least one orifice comprises a region of thickness greater than that of the remaining area, the region having a number of channels within the thickness of that region, these channels leading to the region around the inner orifice opening.

61. The nipple device according to claim 57, wherein at least one orifice has a first inner opening whose ends are fluidly connected to the differential pressure measurement module, and a second outer opening having a larger diameter.
